(19) United States
(12) Patent Application Publication
(10) Pub. No.: US 2011/0086389 A1
(43) Pub. Date: Apr. 14, 2011

(54) Compositions and Methods for Expressing In-Frame Multimeric Proteins

(76) Inventors: James Oliver Dolly, Dublin (IE); Oleg Shamotienko, London (GB)

(21) Appl. No.: 12/915,333

(22) Filed: Oct. 29, 2010

Related U.S. Application Data
(63) Continuation-in-part of application No. PCT/EP90/55304, filed on Apr. 30, 2009.
(60) Provisional application No. 61/126,215, filed on Apr. 30, 2008.

(51) Int. Cl. C12P 21/06; C40B 40/06

(52) U.S. Cl. 435/69.1; 506/16

(57) ABSTRACT

The present invention concerns methods and compositions for making and using multimeric, single chain proteins of a defined structure. In various embodiments the invention involves a bank or collection of one or more monomer nucleic acid species with each such nucleic acid species encoding a protein monomer subunit species and divided into separate subspecies, each bearing a distinct pair of position-specific subcloning restriction endonuclease recognition sites for cassette style cloning into an expression vector and making multimeric, single chain proteins of a defined structure.

### CROSS REFERENCE TO RELATED APPLICATIONS

This application is a continuation-in-part of International Patent Application PCT/EP2009/055304, filed Apr. 30, 2009, which claimed priority to U.S. Provisional application Ser. No. 61/126,215 filed Apr. 30, 2008. The disclosure of each of these documents is expressly hereby incorporated by reference in its entirety.

### BACKGROUND OF THE INVENTION

The nervous system is a feature of all chordates, including mammals, as well as other relatively modern organisms, from worms and slugs to human beings. The nervous system is responsible for sensory, autonomic and motor functions as well as for cognitive functions found in higher animals. The nervous system generally comprises a brain or central processing nerve bundle (ganglion), and various nerves (including other ganglia) or nerve cells which carry sensory information from the body back to the brain and instructions from the brain to the various organs and tissues of the body.

The nerve cell or neuron comprises the basic unit of nerve tissue. The neuron generates and/or conveys information by means of electrical impulses generated and transmitted by the neuron in response to external stimuli, either originating from another nerve cell or from signals in the extracellular milieu. These electrical impulses are possible due to the existence of chemical and electrical gradients formed due to differences in the amounts of charged ions in the cytoplasm of the neuron as compared to the extracellular medium. The gradients are maintained in part by the cell membrane, which is comprised of a lipid bilayer, which provides a barrier preventing facile diffusion of charged ions and molecules between the interior and exterior of the cell, as well as an electrical insulation between the two. The electrical potential difference across the cell membrane is termed the membrane potential. Every living cell has a membrane potential. However nerve cells (and related muscle and gland cells) are thought to be unique in generating and responding to brief changes in the membrane potential as the basis for electrical signaling.

The ions existing within nerve cells comprise organic ions (such as charged peptides and amino acids) and inorganic ions. The main intracellular cation is potassium ion (K⁺). Inorganic anions include chloride (Cl⁻), phosphate (PO₄⁻⁻), and sulfate (SO₄⁻⁻). The main extracellular cation is Na⁺ and the main extracellular anion is Cl⁻. Thus, the electrical activity of nerve cells is derived from the unequal distribution of ions across the cell membrane and the membranes changing permabilities to these ions.

The chemical gradient creates a chemical force tending to move each ion as necessary to achieve homogeneity with respect to that ion on both sides of the cell membrane; while the amount of diffusion across the membrane is small, given enough time this chemical force would, for example, deplete the cell of the K+ sequestered within the cell despite the relatively high impermeability of the cell membrane.

To prevent leakage of electrical charge from inside the cell, the cell membrane contains a metabolic pump (the Na⁺, K+ pump) that maintains the gradient by active transport, moving K+ within the cell (against its concentration gradient) using energy derived from the hydrolysis of ATP to ADP (due to an integral ATPase activity). The Na⁺, K+ ATPase contains an α-subunit of about 100,000 Daltons coupled to a glycoprotein β-subunit of approximately 45,000 Daltons. The amino acid sequence of the α-subunit is known, and comprises 8 transmembrane regions with a large cytosolic domain between transmembrane regions 4 and 5 which contains the ATP hydrolysis site. Binding sites for Na⁺ and K⁺ have also been identified.

The pump transports two different ions in different directions across the membrane. K⁺ ion is bound on the extracellular surface of the pump. The pump is then dephosphorylated, resulting in an allosteric conformation change and transport of the K⁺ ion into the cytoplasm against the concentration and electrical gradient. In a second step, Na⁺ is bound to the cytoplasmic domain of the Na⁺, K⁺ pump, triggering another conformational change, resulting in phosphorylation of the pump at the ATP binding site and transport of the Na⁺ ion against its concentration gradient to the exterior of the cell.

Although the Na⁺/K⁺ pump is an important mechanism of ion transport across membranes, other mechanisms include passive diffusion, bulk flow, facilitated (carrier-mediated) diffusion, exchange diffusion, co-transport, and metabolic and proton pumps. All these mechanisms contribute to maintenance of the membrane potential in neurons although the influence or precise combination of these mechanisms may be different in different neurons.

The ability of a neuron to “fire” (create or transmit an electrical impulse or “action potential”) involves a transient depolarization of the membrane potential of that cell. A depolarization means that the interior of the neuron becomes less negative (and indeed often becomes positive) relative to the exterior of the cell. For example, in squid axons the interior of the cell becomes almost 50 mV positive. This action potential is dependent upon the presence of sodium ions in the external medium.

Thus, the action potential generally involves a selective and transient change in membrane permeability for sodium, causing an inward rush of positively charged sodium ions down their concentration gradient. The energy for this inward migration of sodium is provided by the electrochemical sodium gradient across the cell membrane. However, the mechanism involves a Na⁺ selective transmembrane channel (the Na⁺ channel) which responds to small changes in membrane potential (small depolarizations) by becoming permeable to Na⁺ ion.

Early work on the Na⁺ channel in the electric eel, Electrophorus electricus, showed the Electrophorus sodium channel to be a single linear polypeptide having a molecular weight of 260,000 to 300,000 Daltons. The amino acid sequence of the sodium channel has been solved and the cDNA comprises a chain of 7230 nucleotides encoding a polypeptide of 1820 amino acids. Analysis of the nature of the amino acid sequence reveals 4 domains (equivalent to linked subunits) of about 300 amino acids each having internal sequence homology. Additionally, there are six transmembrane segments within each of the four domains. A tertiary structure of the polypeptide revealed that the four domains are arranged symmetrically around a central pore or channel. At the resting potential the Na⁺ channel is blocked. When the membrane is depolarized, the channel is unblocked, permitting the ingress of Na⁺ ions from outside. A voltage-sensitive region of the Na⁺ channel (thought to reside in an excess of positively charged residues in transmembrane segment 4) responds to increasing membrane depolarization, presumably through an allosteric change permitting the selective influx of sodium ions. The pore forming region of the Na⁺ channel has a cross section no larger than 0.3-0.5 nm to prevent other, larger ions from entering the cell membrane. A highly hydrophobic loop between transmembrane segments 5 and 6 of each domain is thought to form the face of the pore.

Once the Na⁺ channel has been opened to permit Na⁺ ions to enter the neuron, the channel must be subsequently closed to permit the membrane potential to be restored, and the channel to activate again. An intracellular domain of the sodium channel is implicated in this process and appears to act as an “inactivation gate” able to plug the inside mouth of the pore, thus preventing the passage of Na⁺ ions depending on the voltage across the membrane.

Voltage clamp measurements indicate that Na⁺ conductance is involved in a positive feedback relationship with membrane depolarization. Thus, when the membrane begins to be depolarized, the Na⁺ conductance begins to increase, which depolarizes the membrane even more, and so forth. Thus below a given threshold of initial depolarization the channel will fail to fire, but above this initial threshold the channel will open to Na⁺ and the feedback mechanism will take over in an “all or nothing” fashion.

The voltage gated sodium channel is a protein in which the homologous domains discussed so far are referred to as the “α subunit”; as described, forming the pore or “core” of the channel. The α subunit is capable of forming a voltage gated, sodium selective channel alone. However, often the α subunit is found to be associated in a complex with β subunits which can give rise to altered voltage dependence and cellular localization.

The family of voltage gated sodium channels has nine known α protein members, with amino acid identity >50% in the transmembrane and extracellular loop region of each subunit. The standardized nomenclature for sodium channels is outlined below.

The proteins of these channels are named Nav1.1 through Nav1.9. The gene names are referred to as SCN1A through SCN11A (the SCN6/7A gene is part of the Nax sub-family and has uncertain function). The nucleic acid and protein sequences of these subunits reveal likely evolutionary relationships between these channel subunits. The individual sodium channels are distinguished not only by differences in their sequence but also by their kinetics and expression profiles. Some of these data is summarized in Table 1, below.

TABLE 1

| | | GenBank | | | Associated |
|---|---|---|---|---|---|
| Protein | | Accession | Auxiliary | Expression | Human |
| Name | Gene | Number | Subunits | Profile | Channelopathies |
| Naᵥα1.1 | SCN1A | AB093548 | β¹, β2, β3, β4 | Central | Inherited |
| | | | | neurons and | febrile |
| | | | | cardiac | epilepsy, GEFS |
| | | | | myocytes | and myoclonic |
| | | | | | epilepsy |
| Naᵥα1.2 | SCN2A | AB208888 | β₁, β₂, β₃, β₄ | Central | inherited |
| | | | | neurons | febrile seizures |
| | | | | | and epilepsy |
| Naᵥα1.3 | SCN3A | AF035685 | β₁, β₃ | Central | none known |
| | | | | neurons and | |
| | | | | cardiac | |
| | | | | myocytes | |
| Naᵥα1.4 | SCN4A | U24693 | β₁ | Skeletal | hyperkalemic |
| | | | | muscle | periodic |
| | | | | | paralysis, |
| | | | | | Paramyotonia |
| | | | | | congenita, and |
| | | | | | potassium- |
| | | | | | aggravated |
| | | | | | myotonia |
| Naᵥα1.5 | SCN5A | AJ310893 | β₁, β₂, β₃, β₄ | Central | Long QT |
| | | | | neurons, | Syndrome, |
| | | | | cardiac | Brugada |
| | | | | myocytes | syndrome, and |
| | | | | | idiopathic |
| | | | | | ventricular |
| | | | | | fibrillation |
| Naᵥα1.6 | SCN8A | AB027567 | β₁, β₂ | Central | none known |
| | | | | neurons, | |
| | | | | dorsal root | |
| | | | | ganglia, | |
| | | | | peripheral | |
| | | | | neurons | |
| Naᵥα1.7 | SCN9A | X82835 | β₁, β₂ | Dorsal root | Erythromelalgia |
| | | | | ganglia, | and |
| | | | | sympathetic | Channelopathy- |
| | | | | neurons, | associated |
| | | | | Schwann cells, | insensitivity to |
| | | | | and | pain |
| | | | | neuroendocrine | |
| | | | | cells | |
| Naᵥα1.8 | SCN10A | AF117907 | unknown | Dorsal root | none known |
| | | | | ganglia | |
| Naᵥα1.9 | SCN11A | AF126739 | unknown | Dorsal root | none known |
| | | | | ganglia | |

The currently known β sodium channel subunits are as follows: Naᵥβ1, Naᵥβ2, Naᵥβ3 and Naᵥβ4.

In addition to the Na⁺ channel, neurons (and other cells) have Ca⁺⁺ channels and K⁺ channels. These channels have a high degree of amino acid sequence homology with the sodium channel, and their tertiary structures are very similar as well. Like the sodium channel, the Ca⁺⁺ channel has 4 domains and six transmembrane segments, a voltage-sensor activity in the 4^{th} segment, and a domain that lines the interior of the pore. The calcium channel is generally responsible for “short distance” or localized Ca⁺⁺-mediated impulses related to functions controlled within the cell by Ca⁺⁺ ions.

There are several different kinds of high voltage-gated calcium channels (HVGCCs). They are structurally homologous among varying types; they are all similar, but not structurally identical. In the laboratory, it is possible to tell them apart by studying their physiological roles and/or inhibition by specific toxins. High voltage-gated calcium channels include the neural N-type channel blocked by ω-conotoxinGVIA, the R-type channel (R stands for resistant to the other blockers and toxins) involved in poorly defined processes in the brain, the closely related P/Q-type channel blocked by ω-agatoxins, and the dihydropyridine-sensitive L-type channels responsible for excitation-contraction coupling of skeletal, smooth, and cardiac muscle and for hormone secretion in endocrine cells.

There are at least two broad classes of Ca⁺⁺ channel; In the first type, a relatively small depolarization causes transient activation of the channel. These channels are called “T” (or transient) channels; T channels are used by cells to shape the patterns of impulse discharge into bursts. R-type calcium channels respond to intermediate levels of depolarization. The other type of channel requires a stronger depolaraization but continues to open and close during the sustained depolarization. These channels are called “L” (or long lasting) channels, and are (non-exclusively) involved in the control of secretion of neurotransmitters at neural synapses.

The α1 subunit pore is the primary subunit necessary for channel functioning in the HVGCC, and consists of the characteristic four homologous I-IV domains containing six transmembrane α-helices each. The α1 subunit forms the Ca2+ selective pore, which contains voltage sensing machinery and the drug/toxin binding sites. To date a total of ten α1 subunits have been identified in humans, all sharing significant amino acid sequence homology (about 75% to about 90% homology). These are listed in Table 2, below, where the SEQ ID Nos are given in pairs with the odd numbered SEQ ID Nos comprising the nucleotide sequence and the even numbered SEQ ID Nos comprising the protein sequence for that subunit. These α1 subunits may be found in complex with other auxiliary subunits, such as the α2, β, γ and δ subunits, which help shape the activation and deactivation kinetics, and current amplitude.

TABLE 2

| | | α1 | | | |
|---|---|---|---|---|---|
| | | Subunit | GenBank | | |
| | | (Gene | Accession | Associated | Most Often |
| Type | Voltage | Name) | Number | Subunits | Found In |
| L-type | HVA (high | Cav1.1 | L33798 (SEQ | α₂δ, β, γ | Skeletal |
| calcium | voltage | (CACNA1S) | ID NOS 1-2) | | muscle, bone |
| channel (also | activated) | Cav1.2 | AF070589 | | (osteoblasts), |
| known as | | (CACNA1C) | (SEQ ID NOS | | ventricular |
| “Long- | | | 3-4) | | myocytes |
| Lasting” and | | Cav1.3 | AB209171 | | (responsible |
| “DHP | | (CACNA1D) | (SEQ ID NOS | | for prolonged |
| Receptor”) | | | 5-6) | | action |
| | | Cav1.4 | AA019975 | | potential in |
| | | (CACNA1F) | (SEQ ID NO: | | cardiac cell; |
| | | | 7) | | also termed |
| | | | | | DHP |
| | | | | | receptors), |
| | | | | | dendrites and |
| | | | | | dendritic |
| | | | | | spines of |
| | | | | | cortical |
| | | | | | neurones |
| P-type | HVA (high | Cav2.1 | U79666 (SEQ | α₂δ, β, | Purkinje |
| calcium | voltage | (CACNA1A) | ID NOS 8-9) | possibly γ | neurons in the |
| channel/Q- | activated) | | | | cerebellum/ |
| type calcium | | | | | Cerebellar |
| channel | | | | | granule cells |
| N-type | HVA (high | Cav2.2 | AB209467 | α₂δ/β1, | Throughout the |
| calcium | voltage | (CACNA1B) | (SEQ ID NOS | β3, β4, | brain |
| channel | activated) | | 10-11) | possibly γ | |
| (“Neural”) | | | | | |
| R-type | intermediate | Cav2.3 | AK096563(SEQ | α₂δ, β, | Cerebellar |
| calcium | voltage | (CACNA1E) | ID NO: 12) | possibly γ | granule cells, |
| channel | activated | | | | other neurons |
| T-type | low voltage | Cav3.1 | AC004590(SEQ | | neurons, cells |
| calcium | activated | (CACNA1G) | ID NO: 13) | | that have |
| channel | | CaV3.2 | AL031703(SEQ | | pacemaker |
| (“Transient”) | | (CACNA1H) | ID NOS 14-15) | | activity, bone |
| | | CaV3.3 | AF129133(SEQ | | (osteocytes) |
| | | (CACNA1I) | ID NOS 16-17) | | |

The potassium gradient across the cells membrane is in the opposite direction from the Na and Ca++ gradients. That is, the concentration of K+ ions within the cell is higher than in the extracellular medium. Therefore, the K+ channels are important in stabilization of the membrane to a relatively polarized level and to oppose depolarization.

There are at least 8 major groups of K+ channels and are made up of combinations of dozens of subunits. The potassium channels permits the nerve cell to maintain electrochemical equilibrium in the face of a variety of constant activity, which accounts in large part for their diversity. Two broad classes can be defined by their transmembrane topology: those channel proteins having six transmembrane helices in each subunit and those having two transmembrane helices in each subunit.

Among the potassium channels having six transmembrane regions are included:
a) Voltage-gated potassium channels (Kvα and Kvβ)—ion channels that, like the voltage gated Na+ channel, open or close in response to changes in the transmembrane voltage. The Kvα proteins of the voltage gated K+ channels have 6 transmembrane regions, termed S1-S6 in each subunit. Four Kvα subunits surround a central pore channel, involving the S5 and S6 region of each subunit. The selectivity filter (or P region) comprises a hydrophobic amino acid sequence between the last two transmembrane regions and contains the sequence Gly-Tyr-Gly. The voltage sensor region includes multiple positively charged amino acids in the S4 transmembrane region.
b) Calcium-activated potassium channels (SK and Slo subunit families), which open in response to the presence of calcium ions or other signaling molecules. As with the voltage-gated potassium channels, the calcium activated potassium channels have 6 transmembrane regions, termed S1-S6 in each subunit. Four subunits surround a central pore channel, involving the S5 and S6 region of each subunit. The selectivity filter (or P region) comprises a hydrophobic amino acid sequence between the last two transmembrane regions and contains the sequence Gly-Tyr-Gly.
Potassium channels having two transmembrane region-containing subunits include:
a) Inwardly rectifying potassium channels (Kᵢᵣ); These channels may be open at all membrane potentials, but favor the influx, rather than the efflux of K+ ions. Among Kir channels, each subunit has two transmembrane regions. The channel protein is a tetramer of four subunits surround a single pore. As with the voltage-gated channel, each protein may be a homotetramer or a heterotetramer.
b) Tandem pore domain potassium channels (K2P), which are constitutively open or possess high basal activation, such as the “resting potassium channels” or “leak channels” that set the negative membrane potential of neurons. When open, they allow potassium ions to cross the membrane at a rate that is nearly as fast as their diffusion through bulk water. These channels have subunits that are tandem pairs of two K+ channel sequences (most K2P channels are made up of Kᵢᵣ-like amino acid sequences, but a few are known in which a Kv-type subunit and a Kᵢᵣ-type subunit are linked in tandem.

All K channels display a “signature sequence” between the two most carboxy-terminal transmembrane helices, which reads (from amino to carboxy terminus) with minor variations, TMxTVGYG (SEQ ID NO: 18) wherein x is any amino acid.

Alpha subunits form the actual conductance pore of the potassium channel. Based on sequence homology of the hydrophobic transmembrane cores, the alpha subunits of voltage-gated potassium channels have been grouped into 12 classes labeled Kv1-Kv12. The following is a non-exclusive list of 40 known human voltage-gated potassium channel alpha subunits grouped first according to function and then subgrouped according to the Kv sequence homology classification scheme, where the SEQ ID Nos are given in pairs with the odd numbered SEQ ID Nos comprising the nucleotide sequence, and the even numbered SEQ ID Nos comprising the protein sequence for that subunit (except for SEQ ID NOs 113-117, which are as indicated). Gene names are given in parentheses next to the GenBank Accession numbers):

### Delayed Rectifier

Non-Inactivating

Kvα1.x—Shaker-related:

Kv1.1 (KCNA1)(GenBank Accession No L02750 (SEQ ID NOS 19-20)),

Kv1.2 (KCNA2), (GenBank Accession No L02752 (SEQ ID NOS 21-22))

Kv1.3 (KCNA3), (GenBank Accession No L23499 (SEQ ID NOS 23-24))

Kv1.4 (KCNA4), (GenBank Accession No M55514 (SEQ ID NOS 25-26))

Kv1.5 (KCNA5), (GenBank Accession No M83254 (SEQ ID NOS 27-28))

Kv1.6 (KCNA6), (GenBank Accession No X17622 (SEQ ID NOS 29-30))

Kv1.7 (KCNA7), (GenBank Accession No AF315818 (SEQ ID NOS 31-32))

Kv1.8 (KCNA10) (GenBank Accession No U96110 (SEQ ID NOS 33-34))

Kvα2.x—Shab-related:

Kv2.1 (KCNB1), (GenBank Accession No. AF026005 (SEQ ID NOS 35-36))

Kv2.2 (KCNB2) (GenBank Accession No. U69962 (SEQ ID NOS 37-38))

Kvα3.x—Shaw-related:

Kv3.1 (KCNC1), (GenBank Accession No M96747 (SEQ ID NOS 39-40))

Kv3.2 (KCNC2) (GenBank Accession No AF268896 (SEQ ID NOS 41-42))

### Kvα7.x:

Kv7.1; KvLQT1 (KCNQ1)(GenBank Accession No AF000571 (SEQ ID NOS 43-44))

Kv7.2 (KCNQ2), (GenBank Accession No AF268896 (SEQ ID NOS 41-42))

Kv7.3 (KCNQ3), (GenBank Accession No AB208890 (SEQ ID NOS 45-46))

Kv7.4 (KCNQ4), (GenBank Accession No AF105202 (SEQ ID NOS 47-48))

Kv7.5 (KCNQ5) (GenBank Accession No AF202977 (SEQ ID NOS 49-50))

### Kvα10.x:

Kv10.1 (KCNH1) (GenBank Accession No AJ001366 (SEQ ID NOS 51-52))

### A-Type Potassium Channel

Rapidly Inactivating

Kvα3.x—Shaw-related:

Kv3.3 (KCNC3), (GenBank Accession No AB208930 (SEQ ID NOS 53-54))

Kv3.4 (KCNC4), (GenBank Accession No BC101769 (SEQ ID NOS 55-56))

Kvα4.x—Shal-related:

Kv4.1 (KCND1), (GenBank Accession No AF166003 (SEQ ID NOS 57-58))

Kv4.2 (KCND2), (GenBank Accession No AJ010969 (SEQ ID NOS 59-60))

Kv4.3 (KCND3), (GenBank Accession No AF048713 (SEQ ID NOS 61-62))

### Outward-Rectifying

### Kvα10.x:

Kv10.2 (KCNH5) (GenBank Accession No AF472412 (SEQ ID NOS 125-126))

### Inward-Rectifying

Kvα11.x—ether-a-go-go potassium channels:

Kv11.1 (KCNH2)—hERG, (GenBank Accession No U04270 (SEQ ID NOS 63-64))

Kv11.2 (KCNH6), (GenBank Accession No AF311913 (SEQ ID NOS 65-66))

Kv11.3 (KCNH7) (GenBank Accession No AF032897 (SEQ ID NOS 67-68))

### Slowly Activating

### Kvα12.x:

Kv12.1 (KCNH8), (GenBank Accession No AY053503 (SEQ ID NOS 69-70))

Kv12.2 (KCNH3), (GenBank Accession No AB022696 (SEQ ID NOS 71-72))

Kv12.3 (KCNH4) (GenBank Accession No AB022698 (SEQ ID NOS 73-74))

### Modifier/Silencer

Unable to form functional channels as homotetramers but instead heterotetramerize with Kvα2 family members to form conductive channels.

### Kvα5.x:

Kv5.1 (KCNF1) (GenBank Accession No AF033382 (SEQ ID NOS 75-76))

### Kvα6.x:

Kv6.1 (KCNG1), (GenBank Accession No AF033383 (SEQ ID NOS 77-78))

Kv6.2 (KCNG2), (GenBank Accession No AJ011021 (SEQ ID NOS 79-80))

Kv6.3 (KCNG3), (GenBank Accession No AB070604 (SEQ ID NOS 81-82))

Kv6.4 (KCNG4) (GenBank Accession No AF348984 (SEQ ID NOS 83-84))

### Kvα8.x:

Kv8.1 (KCNV1), (GenBank Accession No AF167082 (SEQ ID NOS 85-86))

Kv8.2 (KCNV2) (GenBank Accession No AF348983 (SEQ ID NOS 87-88))

### Kvα9.x:

Kv9.1 (KCNS1), (GenBank Accession No AF043473 (SEQ ID NOS 89-90))

Kv9.2 (KCNS2), (GenBank Accession No AB032970 (SEQ ID NOS 91-92))

Kv9.3 (KCNS3) (GenBank Accession No AF043472 (SEQ ID NOS 93-94))

### Beta Subunits

Beta subunits are auxiliary proteins which associate with alpha subunits in a α4β4 stoichiometry. These subunits do not conduct current on their own but rather modulate the activity of Kv channels.

### Kvβ1 (KCNAB1) GenBank Accession No U33428 (SEQ ID NOS 95-96))

* Kvβ2 (KCNAB2) GenBank Accession No U33429 (SEQ ID NOS 97-98))

* Kvβ3 (KCNAB3) GenBank Accession No AF016411 (SEQ ID NOS 99-100))

* minK (KCNE1) GenBank Accession No L28168 (SEQ ID NOS 101-102))

* MiRP1 (KCNE2) GenBank Accession No AF071002 (SEQ ID NOS 103-104))

* MiRP2 (KCNE3) GenBank Accession No AF076531 (SEQ ID NOS 105-106))

* MiRP3 (KCNE4) GenBank Accession No AY065987 (SEQ ID NOS 107-108))

* KCNE1-like (KCNE1L) GenBank Accession No AJ012743 (SEQ ID NOS 109-110))

* KCNIP1 (KCNIP1) GenBank Accession No AF199597 (SEQ ID NOS 111-112))

* KCNIP2 (KCNIP2) GenBank Accession No Q9NS61 (SEQ ID NOS 113) (this is solely the amino acid sequence)

* KCNIP3 (KCNIP3) GenBank Accession No AF199599 (SEQ ID NOS 114-115) (these are nucleotide and protein sequences, respectively)

* KCNIP4 (KCNIP4) GenBank Accession No AF453244 (SEQ ID NOS 116-117) (these are nucleotide and protein sequences, respectively).

Proteins minK and MiRP1 are putative hERG beta subunits.

Each and every patent, patent application, publication, website or database reference mentioned in this specification, including, without limitation, gene names, accession numbers and all information associated therewith, are hereby incorporated by reference herein in their entirety as part of this patent application.

### SUMMARY OF THE INVENTION

The present invention includes a general strategy and practical approach for the preparation of nucleic acid constructs and mutimeric single chain proteins using recombinant DNA methods. Expressed protein constructs comprise concatenated monomeric subunits linked in the same open reading frame and having pre-determined subunit position and stoichiometries.

In particular, methods of the present invention have the advantage of providing a “cassette” method for construction of substantially homogeneous preparations of specific multi-subunit proteins. These methods may involve the preparation of a bank or collection of nucleic acids encoding monomeric polypeptide subunits of the multimeric protein or family of multimeric proteins of interest. Each nucleic acid fragment encodes a monomeric polypeptide subunit and is altered to contain restriction endonuclease sites or “half sites” (i.e., sites containing either the 5′ or 3′ portions of a restriction endonuclease site) at its 5′ or 3′ terminus, thus permitting its assembly into a nucleic acid construct capable of expressing a specific multimeric protein of interest.

Multi-subunit proteins may be of interest in, for example, studying various proteins and protein complexes comprising polypeptide subunits. Additionally, such proteins may be used as targets for the screening of potential therapeutic agents.

Non-limiting examples of such proteins and protein complexes may comprise: Kᵥ voltage-activated K⁺ channels, calcium activated potassium channels, ATP-sensitive K⁺ channels, inward-rectified K⁺ channels, ligand-gated ion channels, such as the superfamily of ionotropic Cys-loop receptors (including cationic receptors such as the nicotinic acetycholine receptor, the 5HT₃ receptor and the serotonin receptor, and anionic receptors such as the glycine receptor, the GABA_{A} and GABA_{B} receptors), glutamate receptors, such as the NMDA (N-methyl-D-aspartate receptor, the AMPA (α-amino-3-hydroxy-5-methyl-4-isoxazole priopionic acid) receptors and the kainate receptors, ATP-gated receptors containing P2X₁-P2X₆ subunits.

Also included within the scope of the invention are methods involving the preparation of a bank or collection of nucleic acids encoding monomeric polypeptide subunits of the multimeric protein or family of multimeric proteins of interest, wherein each nucleic acid fragment encodes a monomeric polypeptide subunit and is altered to contain restriction endonuclease sites or “half sites” (i.e., sites containing either the 5′ or 3′ portions of a restriction endonuclease site) at its 5′ or 3′ terminus. According to this method, the identity of the restriction endonuclease sites at the 3′ and 5′ terminus of a given nucleic acid fragment is different depending upon the desired position of the polypeptide subunit in the concatenated multi-subunit protein. Thus, a nucleic acid encoding a given polypeptide subunit may be altered to have different pairs of restriction endonuclease sites or half sites for each desired position in the multimeric protein. Very preferably these sites do not occur within the coding sequence of the nucleic acid fragment.

Thus, also included within the scope of the present invention is a method for the preparation of a nucleic acid expression system for the production of proteins encoding two or more distinct amino acid sequences, whose position within the protein is dictated by a distinct pair of restriction endonuclease sites (or half sites) at the 5′ and 3′ termini.

Also included in the present invention is a method for the preparation of a single chain polypeptide comprising a mammalian ion channel selected from the group consisting of a sodium ion channel, a calcium ion channel and a potassium ion channel, wherein the ion channel comprises two or more subunits. Preferably the ion channel is a human ion channel. The invention also includes nucleic acid constructs for the expression of multimeric proteins comprising two or more ion channel subunits, and cells expressing such multi-subunit proteins.

The invention also includes method for the preparation of nucleic acid expression systems, and the nucleic acid expression system itself, containing nucleic acids encoding a concatenated multimeric protein comprising at least one mammalian Kv voltage-activated potassium channel subunit from a potassium channel family selected from the group consisting of Kvα1, Kvα2, Kvα3, Kvα4, Kvα5, Kvα6, Kvα7, Kvα8, Kvα9, Kvα10, Kvα11 and Kv12. In particular, the subunit may be selected from the group consisting of Kv1.1, Kv1.2, Kv1.3, Kv1.4, Kv1.5, Kv1.6, Kv1.7, Kv1.8, Kv2.1, Kv2.2, Kv3.1, Kv3.2, Kv3.3, Kv3.4, Kv4.1, Kv4.2, Kv4.3, Kv5.1, Kv6.1, Kv6.2, Kv6.3, Kv6.4, Kv7.1, Kv7.2, Kv7.3, Kv7.4, Kv7.5, Kv8.1, Kv8.2, Kv9.1, Kv9.2, Kv9.3, Kv10.1, Kv10.2, Kv11.1, Kv11.2, Kv11.3, Kv12.1, Kv12.2, and Kv12.3. In a preferred embodiment the amino acid sequence of the potassium channel subunit is from 74% homologous to 100% homologous, or at least 75%, or at least 80%, or at least 85%, or at least 90%, or at least 95%, or at least 98% or at least 99% homologous to a human potassium channel subunit. It will be understood that the range of homology from 75% homologous to 100% homologous specifically discloses, and is intended to specifically disclose, each and every degree of homology, expressed as an integer or a fraction, within this range.

By “amino acid sequence homology” is meant the degree of similarity of contiguous amino acid sequences in two designated regions of one or more protein or polypeptide molecules when the sequences are compared.

By “nucleotide sequence homology” is meant the degree of similarity of contiguous nucleotide sequences in two designated regions of one or more nucleic acid molecules when the sequences are compared.

Preferably, the method for the preparation of nucleic acid expression systems, and the nucleic acid expression system itself, containing nucleic acids encoding a concatenated multimeric protein comprising at least one mammalian Kv voltage-activated potassium channel subunits independently selected from the group consisting of Kv1.1, Kv1.2, Kv1.3, Kv1.4, Kv1.5, Kv1.6, Kv1.7, and Kv1.8 potassium channel subunits. The amino acid sequence of at least one mammalian Kv voltage-activated potassium channel subunit is preferably from 75% to 100% homologous to the human amino acid sequence of the analogous potassium channel subunit. Similarly, the nucleic acid sequence encoding at least one mammalian Kv voltage-activated potassium channel subunit is preferably from 75% to 100% homologous to the nucleic acid sequence of the counterpart human potassium channel subunit. It will be understood that the range of homology from 75% homologous to 100% homologous specifically discloses, and is intended to specifically disclose, each and every degree of homology, expressed as an integer or a fraction, within this range.

Preferably, although not invariably, at least 2 or at least 3 or at least 4 subunits of said multimeric protein are independently selected from the group consisting of Kv1.1, Kv1.2, Kv1.3, Kv1.4, Kv1.5, Kv1.6, Kv1.7, and Kv1.8 potassium channel subunits.

The present invention also includes methods of identifying a modulator of a multimeric protein of defined structure comprising producing a substantially homogenous preparation of a multimeric protein having a predetermined identity and order of monomer subunits and a detectable biological activity, contacting said multimeric protein with a test compound, detecting the biological activity of the multimeric protein, if any, and comparing the biological activity of the multimeric protein in the presence and absence of said test compound, wherein a difference in the biological activity of the multimeric protein in the presence and absence of the test compound indicates that the test compound is a modulator of said multimeric protein.

The present invention also includes methods of identifying and assigning molecular structures to native potassium channels comprising producing substantially homogenous preparations of various multimeric proteins having predetermined molecular structure and order of monomer subunits and a set of defined biological and biophysical properties, including for example and without limitation, sensitivity or lack thereof to test compounds, degree of sensitivity to test compounds, determining biological and biophysical properties of native potassium channels, comparing the biological and biophysical properties of the multimeric proteins of predetermined molecular structure and order of monomer subunits and those of the native potassium channels, wherein similarities in the biological properties of native potassium channels and multimeric proteins predetermined molecular structure and order of monomer subunits indicates similarities in molecular structures.

It will be understood that by “biological activity” is meant a test of, for example, a change in protein conformation, a change in the enzymatic activity of the protein, a change in chemical reactivity of the protein, a change in permeability of a cell membrane into which the protein is integrated, a change in conductance of electrical charge, a change in ligand binding characteristics and the like, conducted either in vivo or in vitro.

A “test compound” may be a discrete molecule or macromolecule, be a part of a larger molecule or group of molecules, or comprise a complex of molecules.

The invention also includes automated methods for carrying out the identification of a modulator of a multimeric protein of defined structure as described above wherein the contacting and comparing steps are performed using a computerized systems, such as a robotic system, in which a plurality of test compounds are contacted, either separately or in pools, with the multimeric protein. In such a system the detection of the biological activity of the multimeric protein (in the presence or absence of a test compound) or lack thereof and/or comparison steps are stored in a computer.

Preferably, but not exclusively, the multimeric protein of defined structure may be tested for ion channel activity, such as, without limitation, sodium, calcium or potassium channel activation or deactivation activity, conductance, kinetics of conductance, toxin inactivation susceptibility, and the like. In another test of biological activity, the multimeric protein of defined structure may be tested for ligand binding avidity, phosphorylation/dephosphorylation reactions, other enzymatic activities, or conformational changes such as those associated with cell surface receptors.

Compounds having a modulating activity on the biological activities of such multimeric proteins may include agonists, inverse agonists and/or antagonists of the naturally occurring version of the multimeric protein. An agonist stimulates the tested biological activity of the multimeric protein and an antagonist reduces or blocks the tested biological activity of the multimeric protein. An inverse agonist actually causes a reduction in the biological activity of the multimeric protein beyond that seen in the presence of an efficient antagonist or “blocker”. In other words, the inverse agonist is capable of stimulating an activity counter to that of the tested biological activity of the multimeric protein in the absence of the test compound.

Consistent with the present invention, a nucleic acid expression system for the expression of a multimeric protein may be made by first constructing a bank or collection of nucleic acid fragments encoding one or more subunits of a multimeric protein. The nucleic acid fragments are altered to contain recognition sites (or half sites) for different restriction endonucleases, one at or near (but preferably not within) the 5′ terminus of the coding sequence, and one at or near (but preferably not within) the 3′ termini of the coding sequence. The identity of the restriction sites determine the position of the subunit in the expressed multimeric protein.

An example of a restriction endonuclease recognition site is the nucleotide sequence recognized by the restriction endonuclease Eco RI. Upon cleavage, a 3′ (by convention referring to the strand written left to right from 5′ to 3′) terminus is created having a 5′ overhang, and a 5′ terminus is also created having a 5′ overhang. The portions of a restriction endonuclease recognition sequence remaining on the 5′ and 3′ termini following cleavage are referred to herein as “half sites”.

It will therefore be apparent that when a restriction endonuclease half site is located at the 5′ terminus of a nucleic acid fragment, the half site will be the portion of the recognition sequence located to the 3′ side of the position of endonuclease cleavage, whereas a half site located at the 3′ terminus of a nucleic acid fragment will be the portion of the recognition sequence located to the 5′ side of the cleavage point.

Thus, a 3′ half site of a given restriction endonuclease may be ligated to a 5′ half site for the same restriction endonuclease (or one leaving identical overhanging ends) and the resulting ligated nucleic acid will contain a regenerated recognition sequence, which may subsequently be cleaved upon exposure to the restriction endonuclease.

In the present invention, nucleic acid fragments encoding monomer subunits are very preferably altered to contain nucleotide sequences such that a pair of recognition sites (or half sites) for different restriction endonucleases is generated; one at or near (but preferably not within) the 5′ terminus of the coding sequence, and one at or near (but preferably not within) the 3′ terminus of the coding sequence. Preferably, the identities of the restriction sites or half sites is assigned on the basis of the desired position of the monomer subunit within the multimeric protein.

As a result, it may be desirable for each nucleic acid fragment encoding a given monomer subunit to contain a different pair of restriction sites or half sites for each monomer position in the multimeric protein. Also, the identity of the restriction sites or half sites preferably correspond to the defined position for each monomer in the resulting multimeric protein. Thus, the specific pair of restriction endonuclease sites or half sites at the 5′ and 3′ termini of all monomeric subunit-encoding nucleic acid fragments will preferably be the same for each desired monomer position in the resulting multimeric protein.

### BRIEF DESCRIPTION OF THE DRAWINGS

FIG. 1 is a schematic representation of the construction of an expression vector comprising four distinct nucleic acid fragments flanked by restriction sites for the expression of multimeric proteins. Also shown are the subunit position numbers in the multimeric protein.

FIG. 2A is an agarose gel electrophoretogram of intermediate Kv1.1 constructs to verify the assembly of the subunit monomers in the desired position of the expression vector.

FIG. 2B is an agarose gel electrophoretogram showing the result of restriction digests of the Kv1.1(#1)-Kv1.1(#2)-Kv1.2(#3)-Kv1.2(#4)-pIRES2-EGFP plasmid.

FIG. 3 shows the result of a Western blot of the expression products of plasmids Kv1.2(#4)-pIRES2-EGFP, Kv1.2(#3)-Kv1.2(#4)-pIRES2-EGFP, Kv1.1(#3)-Kv1.2(#4)-pIRES2-EGFP, Kv1.1(#2)-Kv1.2(#3)-Kv1.2(#4)-pIRES2-EGFP, and Kv1.1(#1)-Kv1.1(#2)-Kv1.2(#3)-Kv1.2(#4)-pIRES2-EGFP.

FIG. 4A shows the results of an assay in which the Kv1.1-1.1-1.2-1.2 tetramer channel is expressed in cells and displayed on membranes to form fully functional potassium channels. Shown is affinity binding of radiolabled DTX, an inhibitor of Kv1.x channel.

FIG. 4B shows the results of a competition binding assay in which the Kv1.1-1.1-1.2-1.2 tetramer channel is expressed in cells and displayed on membranes to form fully functional potassium channels. The membranes are inculbated with 2.5 μM 125I-αDTX and various concentrations of the indicated toxins αDTX (closed circle), DTXₖ (open circle), or TsTx (tityustoxin-Kₐ). The figure shows the relative strengths of specific binding.

FIG. 4C shows the results of a competition binding assay in which the Kv1.1-1.1-1.2-1.2 tetramer channel is expressed in cells and displayed on membranes to form fully functional potassium channels. The membranes are inculbated with 2.5 μM ¹²⁵I-αDTX and various concentrations of ShK and its derivative ShK-Dap²². The figure shows the relative strengths of specific binding.

FIG. 5 shows immunoblots of lysates from HEK 293 cells transfected with plasmids expressing the indicated tetramer constructs. FIG. 6 shows a Western blot of a preparation of intact HEK 293 cells expressing tetramers Kv 1.1-1.1-1.2-1.2, 1.1-1.2-1.1-1.2, 1.2-1.2-1.1-1.1 and 1.2-1.1-1.2-1.1.

FIGS. 7A and 7B is an agarose gel electrophoretogram showing the result of restriction digests of the (a) Kv1.1(#1)-Kv1.1(#2)-Kv1.2(#3)-Kv1.2(#4)-pIRES2-EGFP plasmid; (b) Kv1.1(#1)-Kv1.2(#2)-Kv1.1(#3)-Kv1.2(#4)-pIRES2-EGFP plasmid; (c) Kv1.2(#1)-Kv1.2(#2)-Kv1.1(#3)-Kv1.1(#4)-pIRES2-EGFP plasmid; and (d) Kv1.2(#1)-Kv1.1(#2)-Kv1.2(#3)-Kv1.1(#4)-pIRES2-EGFP plasmid.

FIG. 8 shows fluorescent micrographs depicting surface expression patterns in transfected COS-7 cells expressing fully functional hetero-tetrameric Kv1 channels.

FIGS. 9 and 10 show whole-cell voltage-clamped recordings of concatenated Kv1 heteromers Kv 1.1-1.1-1.2-1.2, 1.4-1.6-1.1-1.2 and 1.4-1.6-1.3-1.2 in corresponding HEK-293 transfected cells and the sensitivities of the potassium channel currents mediated by Kv 1.1-1.1-1.2-1.2 Kv 1.4-1.6-1.1-1.2 and Kv1.4-1.6-1.3-1.2 to TEA, αDTX, DTXₖ, 4-aminopyridine (4AP), TsTX and ShK-Dap²².

FIGS. 11 and 12 show whole-cell voltage-clamped recordings of HEK 293 transfected with concatenated Kv1 heteromers Kv 1.1-1.1-1.2-1.2, 1.4-1.6-1.1-1.2 and 1.4-1.6-1.3-1.2 and the sensitivities of the potassium channel currents mediated by these concatamers to TEA and/or AGTX1 and TsTX-Kα.

FIG. 13 is a non-exclusive illustration of a scheme according to the present invention showing a generalized cassette method for the construction of a desired polymeric protein of defined subunit order.

### DETAILED DESCRIPTION OF THE INVENTION

The present invention includes a general strategy and practical approach for the preparation of nucleic acid constructs and multimeric single chain proteins using recombinant DNA methods. Expressed protein constructs comprise concatenated monomeric subunits linked in the same open reading frame and having pre-determined subunit position and stoichiometries.

In various embodiments of the methods, systems and compositions of the invention an important feature includes a bank or collection of one or more monomer nucleic acid species with each such nucleic acid species encoding a protein monomer subunit species and divided into separate subspecies each bearing a distinct pair of position-specific subcloning restriction endonuclease recognition sites. The subcloning restriction endonuclease recognition sites are introduced by joining the 5′ and 3′ ends of each nucleic acid subspecies with a position-specific designated nucleic acid linker pair. The position-specific designated nucleic acid linker pairs joined to the subspecies of each nucleic acid species are the same for each desired position of the protein monomer subunit species in an expressed protein polymer. Thus, there will be as many designated linker pairs as monomer units in the expressed protein polymer. The set of position-specific linkers for construction of a given polymer is referred to herein as a “linker set”.

Each linker pair is structured to comprise a 5′ designated linker having a 5′ subcloning restriction endonuclease recognition site different from that of the 5′ designated linker of any other designated linker pair of the linker set; and a 3′ designated linker having a 3′ subcloning restriction endonuclease recognition site different from that of any other 3′ designated linker of any other designated linker pair of the linker set.

Linkers (including primers) preferably contain a Xenpus β globin UTR region to aid in expression of the polymeric protein.

Furthermore, each linker set is structured so that the 3′ subcloning restriction endonuclease recognition site of the first designated linker pair (the linker pair designating “Position 1” of the expressed protein) is the same as the 5′ subcloning restriction endonuclease recognition site of the second designated linker pair and the 3′ subcloning restriction endonuclease recognition site of the second designated linker pair (the linker pair designating “Position 2” of the expressed protein) is the same as the 5′ subcloning restriction endonuclease recognition site of the third designated linker pair, and so forth.

Cleavage of the linker pairs with the appropriate restriction endonuclease results in the corresponding “half sites” on each end of the nucleic acid subspecies, available to anneal with other nucleic acids appropriately cleaved with the same or isoschizomeric restriction endonucleases. The resulting nucleic acid constructs are “position-specific” nucleic acid subspecies.

When constructing expression vectors for the expression of a desired polymeric protein, the appropriate position specific nucleic acid subspecies for each position of the desired polymeric protein are annealed, joined, and subcloned into an appropriate cloning site of an expression vector. The vector is then introduced into a suitable host cell to produce a polymeric protein comprising a defined order of specific monomer subunits.

Thus, in one embodiment the present invention is drawn to a collection of nucleic acids comprising one or more nucleic acid monomer subunit species separately joined to each of first, second, third and fourth designated nucleic acid linker pairs of to make first, second, third, and fourth nucleic acid monomer subunit subspecies, wherein each designated nucleic acid linker pair comprises a 3′ subcloning restriction endonuclease recognition site and a 5′ subcloning restriction endonuclease recognition site, wherein;

a) the 3′ subcloning restriction endonuclease recognition site of the first designated linker pair is the same as the 5′ subcloning restriction endonuclease recognition site of the second designated linker pair;

b) the 3′ subcloning restriction endonuclease recognition site of the second designated linker pair is the same as the 5′ subcloning restriction endonuclease recognition site of the third designated linker pair;

c) the 3′ subcloning restriction endonuclease recognition site of the third designated linker pair is the same as the 5′ subcloning restriction endonuclease recognition site of the fourth designated linker pair; and wherein the first, second, third, and fourth nucleic acid monomer subunit subspecies are structured to maintain a common open reading frame when ligated together in the sequence, in the direction from 5′ to 3′, first, second, third, and fourth nucleic acid monomer subunit subspecies.

Those of skill in the art recognize that the polymeric proteins of the present invention (and thus nucleic acid cassettes encoding them) need not be limited to tetrameric proteins or corresponding cassettes, but can be comprised of as many subunits as there are position-specific linker pairs in the linker set. By “linker” is also included PCR primers that are incorporated within an amplicon.

In another embodiment the invention is drawn to a method of making a polymeric protein having a N-terminus and a C-terminus and a defined order of specific monomer subunits covalently linked, comprising:

a) amplifying one or more monomer nucleic acid species encoding a monomer subunit species to be contained in the polymeric protein;

b) creating a linker set comprising first, second, third and fourth designated nucleic acid linker pairs; wherein
1) each designated linker pair comprises a 5′ designated linker having a 5′ subcloning restriction endonuclease recognition site different from that of the 5′ designated linker of any other designated linker pair of said linker set;
2) a 3′ designated linker having a 3′ subcloning restriction endonuclease recognition site different from that of any other 3′ designated linker of any other designated linker pair of said linker set;
3) wherein each of the first, second, third, and fourth designated linker pairs is structured to correlate with a first, second, third, and fourth monomer subunit position, respectively, in the tetrameric protein; and
4) wherein
   A) the 3′ subcloning restriction endonuclease recognition site of the first designated linker pair is the same as the 5′ subcloning restriction endonuclease recognition site of the second designated linker pair;
   B) the 3′ subcloning restriction endonuclease recognition site of the second designated linker pair is the same as the 5′ subcloning restriction endonuclease recognition site of the third designated linker pair;
   C) the 3′ subcloning restriction endonuclease recognition site of the third designated linker pair is the same as the 5′ subcloning restriction endonuclease recognition site of the fourth designated linker pair; and

c) individually joining amplified monomer nucleic acid species to 3′ and 5′ designated linkers of each of the first, second, third and fourth designated linker pairs to form a bank of first, second, third, and fourth in-frame nucleic acid monomer species constructs encoding a specific monomer subunit, each said construct having designated restriction site pairs at its 3′ and 5′ end correlating to one of four positions within the polymeric protein,

d) selecting a first, second, third, and fourth nucleic acid monomer species construct encoding the desired monomer subunit for each such position of the polymeric protein,

e) subcloning said first, second, third and fourth nucleic acid monomer species constructs into an expression vector having, in the 5′ to 3′ direction, restriction sites for joining with the 5′ restriction site of each of the first, second, third and fourth nucleic acid monomer species constructs, and a 3′ restriction site for joining with the 3′ end of the fourth nucleic acid monomer species construct, wherein the expression vector is structured to insert each nucleic acid monomer species construct in a common open reading frame, and

f) inserting said expression vector within a suitable host cell,

g) expressing within said host a polypeptide comprising a polymeric protein comprising a defined order of specific monomer subunits.

Again, those of ordinary skill will recognize immediately that this method need not be limited to methods of making tetrameric proteins, but can be applied to the synthesis of proteins comprised of as many subunits as there are position-specific linker pairs in the linker set. By “linker” is also included PCR primers that are incorporated within a nucleic acid amplicon.

In yet another embodiment the invention includes a kit comprising a cassette cloning system for the expression of a concatenated multisubunit protein comprising;

a) a nucleic acid expression vector comprising a multiple cloning site having at least a first restriction nuclease recognition site and a second different nucleic acid recognition site located to the 3′ of the first site; and

b) a nucleic acid collection comprising one or more nucleic acid monomer subunit species separately joined to each of first, second, third and fourth designated nucleic acid linker pairs of to make first, second, third, and fourth nucleic acid monomer subunit subspecies, wherein each designated nucleic acid linker pair comprises a 3′ subcloning restriction endonuclease recognition site and a 5′ subcloning restriction endonuclease recognition site, wherein;

a) the 3′ subcloning restriction endonuclease recognition site of the first designated linker pair is the same as the 5′ subcloning restriction endonuclease recognition site of the second designated linker pair;

b) the 3′ subcloning restriction endonuclease recognition site of the second designated linker pair is the same as the 5′ subcloning restriction endonuclease recognition site of the third designated linker pair;

c) the 3′ subcloning restriction endonuclease recognition site of the third designated linker pair is the same as the 5′ subcloning restriction endonuclease recognition site of the fourth designated linker pair; and wherein the first, second, third, and fourth nucleic acid monomer subunit subspecies are structured to maintain a common open reading frame when ligated together in the sequence, in the direction from 5′ to 3′, first, second, third, and fourth nucleic acid monomer subunit subspecies,

wherein the 5′ subcloning restriction endonuclease recognition site of one nucleic acid monomer subunit subspecies is the same as the first vector restriction endonuclease recognition site, and the 3′ subcloning restriction endonuclease recognition site of another nucleic acid monomer subunit subspecies is the same as the second vector restriction endonuclease recognition site.

Those of skill in the art recognize that kits such as those described may have more of less subunit species than the four described above without departing from the scope of the invention.

The invention also includes nucleic acid constructs for the expression of substantially homogeneous preparations of recombinant multimeric proteins of predetermined structure. By this is meant that the preparations preferably contain less than about 20% or 15% or 10% or 5% by weight of any multimeric protein made from one or more of the same monomer subunits, which protein has an amino acid sequence different from the sequence recombinant multimeric protein. Thus, a substantially homogeneous preparation of a given multimeric protein may include a cell lysate, an in vitro translation reaction mixture, chromatography fractions, (such as ion exchange, reverse phase, and gel exclusion fractions), and the like. Particularly useful is a cassette-based nucleic acid expression system for easily making any of a variety of different multimeric proteins using monomeric subunit-encoding nucleic acid fragments.

The invention is useful for creating any of a variety of monomer-based single chain polypeptides. Such polypeptides may be based, without limitation, upon 1) naturally occurring single chain multimeric proteins (such as the immunoglobulin heavy chains and light chains), 2) multimeric protein complexes in which monomer subunits naturally occur as single chains and associate into a quaternary structure, or 3) non-naturally occurring single chain multimeric proteins.

In certain embodiments the single chain multimeric polypeptides of the present invention aggregate or associate into a tertiary structure functionally similar to the quaternary structure of a naturally occurring multimeric protein complex made from the same or similar subunits.

The invention also includes methods of identifying a modulator of a biological activity possessed by a multimeric protein from a population of compounds, comprising contacting said multimeric protein with a compound under conditions sufficient to detect a change in a biological activity of the multimeric protein in the presence and the absence of the compound, and identifying a compound that causes such a change in said biological activity of said multimeric protein as a modulator of said biological activity.

The Examples that follow comprise specific embodiments of the present invention, which is, however, not limited only to the disclosure of these examples, but is defined solely by the claims that conclude this specification.

### EXAMPLES

To exemplify the present invention, a set of voltage-activated K⁺ channels were made. Voltage-activated K⁺ channels (Kv1) represent a diverse group of sialoglycoprotein complexes consisting of four transmembrane channel-forming α subunits and four cytoplasmically-associated auxiliary β proteins. Their numerous functions include control of neuronal excitability, shaping of action potentials, determining the inter-spike interval and indirectly, modulation of synaptic transmission. Expression of mutant Kv1.X monomeric subunits in vivo can result in severe neurological disorders such as episodic ataxia I and myokymia. Moreover, conditions like multiple sclerosis and Alzheimer's disease are accompanied by changes in the level of expression of Kv1.X channels. Therefore, it is of considerable importance to investigate the fundamental roles served by these channels; this requires recreation of heterologously-expressed complexes mimicking the composition and stoichiometry of their native counterparts.

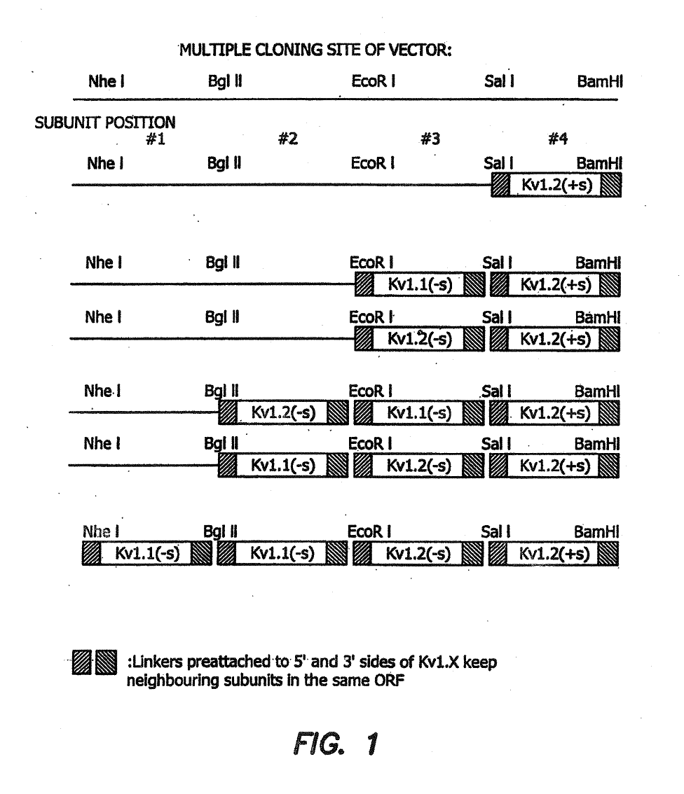

The present examples illustrate the invention by employing two separate rounds of PCR-amplification of Kv1.X genes to generate the PCR-Blunt® plasmid-based bank of all individual Kv1.X monomeric constituents with pre-attached linker regions. Individual subunits from this bank can be directly inserted into pIRES2-EGFP plasmid which is used as an assembling platform for the preparation of any type of Kv1.X concatenated channel construct (outlined in FIG. 1).

In the first stage, PCR products of each individual monomeric Kv1.X gene were generated using Kv1.X-pAKS plasmids as templates and primers based on the 5′ and 3′ terminal coding sequences of the respective Kv1.X genes, with a Xba I flanking site for all forward primers and a Xho I site in all reverse primers. The presence of 5′ Xba 1 and 3′ Xho I restriction sites in Kv1.X PCR products permitted their cloning into the modified pβUT2 plasmid, digested identically. Modification of the original pβUT2 plasmid was performed to delete those sites which were also present in the pIRES2-EGFP plasmid, and which could therefore interfere with the construction methodology.

The second round of PCR-amplification attached identical half-size linker regions to the 5′ and 3′ ends of Kv1.X genes, with simultaneous introduction of flanking restriction sites specific for each assembly position in concatenated oligomers. In assembled oligomeric constructs, half-size linker regions on the left and right hand sides of two neighboring monomeric subunits formed a full-length linker, keeping those two Kv1.X-subunits in the same open reading frame (ORF). Kv1.X-pβUT2 plasmids prepared after the initial PCR were used as templates for the second round, together with primers based not on the sequence of individual Kv1.X-genes but on that of the untranslated regions (UTR) of Xenopus β-globin gene inserted into pβUT2 and flanking its Xba I and Xho I cloning sites. It was shown previously that these UTR regions can serve as a linker for joining two concatenated Kv1.X subunits.

Forward primers corresponded exactly to the last 30 nucleotides of the 5′ UTR up to Xba I cloning site; reverse primers were based on the 30-mer stretch of the 3′ UTR starting downwards from the Xho I cloning site. Since both primers were based on the sequence of UTRs, any type of gene placed between Xba I and Xho I cloning sites of pbUT2 could be amplified using the same pair of primers. Thus, all PCR-products of Kv1.X genes, prepared at this stage, consisted of the full gene sequence with flanking 30-mer pieces of 5′ UTR at their 5′ ends and 3′ UTR DNA at their 3′ ends. They were bluntly cloned into PCR-Blunt® plasmid which allowed creation of a bank of ready-to-use monomers for the assembling of any Kv1.X concatenated complex.

Either pIRES2-EGFP plasmid or pIRES-DsRed were chosen as an assembling platform for the following reasons. Firstly, they contain a sufficient number of cloning restriction sites which could easily accommodate all four channel α subunits, cloned independently one by one. Secondly, presence of either EGFP or DsRed makes these plasmids convenient for monitoring the transfection and expression, using fluorescent microscopy.

Four cloning positions of pIRES2-EGFP plasmid were utilised for assembling the oligomeric constructs. Position #1, corresponding to the first Kv1.X constituent in the resultant tetramer, was situated and utilized between Nhe I and Bgl II sites. Therefore, UTR-specific primers used for constructing the PCR products to be cloned into this position were flanked with Nhe I for forward and Bgl II for reverse primers, respectively. Cloning position #2, relating to the second constituent in the tetramer, resided between Bgl II and Eco RI sites; UTR-specific primers for this position were flanked with these two restriction sites. Cloning position #3, corresponding to the third constituent in the tetramer, was located between EcoR I and Sal I sites; these sites were employed for flanking forward and reverse primers, respectively, in the third group of UTR-specific primers. Finally, cloning position #4, containing the fourth situated constituent in the tetramer (and also containing a stop codon), was between Sal I and BamH I sites; forward primers in the fourth UTR-specific group of primers were flanked with Sal I and reverse with BamH I sites.

Thus, all four groups of UTR-specific primers, based on the same sequences of 5′-UTR for all forward and 3′-UTR for all reverse primers respectively, differed only by position-specific flanking restriction sites. This presence of unique restriction sites for each pair of primers made them actually “position-specific” rather than “subunit-specific”. Since each of the half-linkers consisted of 30 nucleotides and all restriction sites contained 6 nucleotides, every two neighbouring subunits in the assembled oligomers (regardless of their exact position) were separated by the 78-nucleotide stretches of linker regions (including 6 nucleotides for each Xba I and Xho I sites added in the first round of PCR) which kept all of them in the same ORF. Analysis of linker regions showed the presence of relatively low numbers of hydrophobic amino acids and the absence of internal stop codons.

Although the following examples demonstrate a method that begins the assembly of the multimeric expression construct with the fourth position, it is worth noting that assembling of concatenated oligomers can be started from any of the four cloning positions. Similarly, any constituent in the prepared oligomer can be excised and replaced with any other desirable Kv1.X nucleic acid monomer. In this case, assembly was started from the position #4.

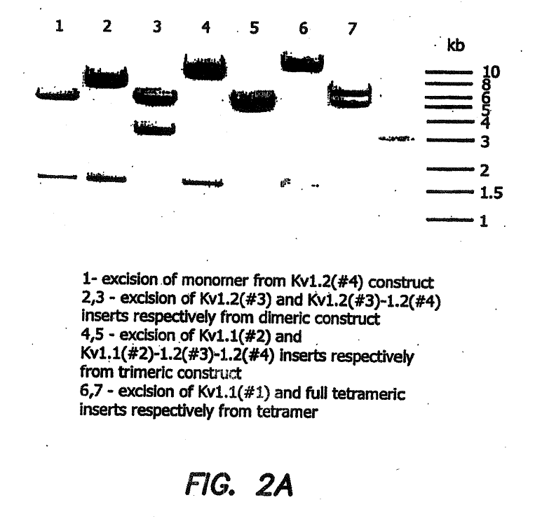

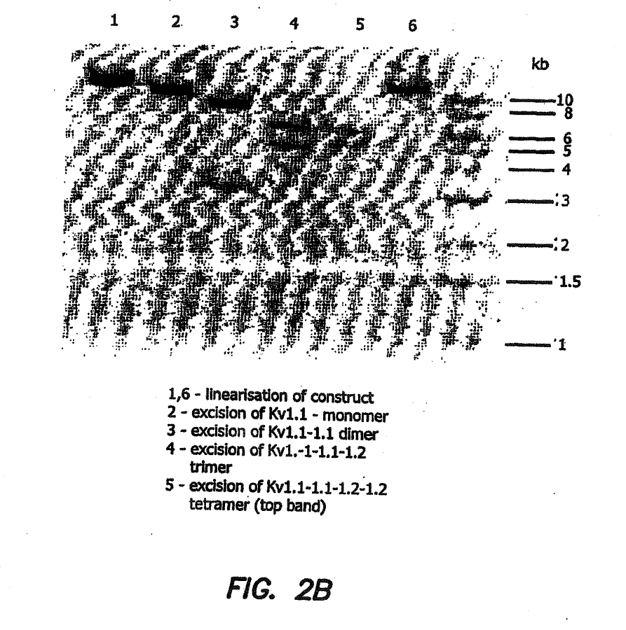

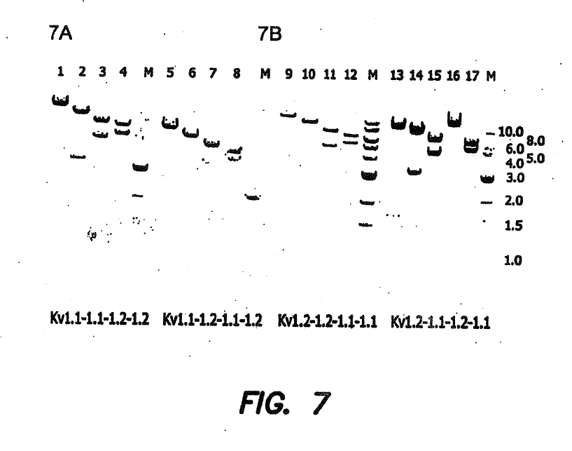

Monomeric Kv1.X1 (+stop-codon) subunit inserted into position #4 itself represents a construct suitable for expression. Insertion of Kv1.X2 into position #2 transforms monomeric construct into expressible Kv1.X2-1.X1 dimer. Consecutive addition of monomers into positions #3 and #4 resulted in the creation of a full tetramer. Constructs prepared at each stage were verified by restriction digestion (See FIGS. 2A, 2B, 7A and 7B).

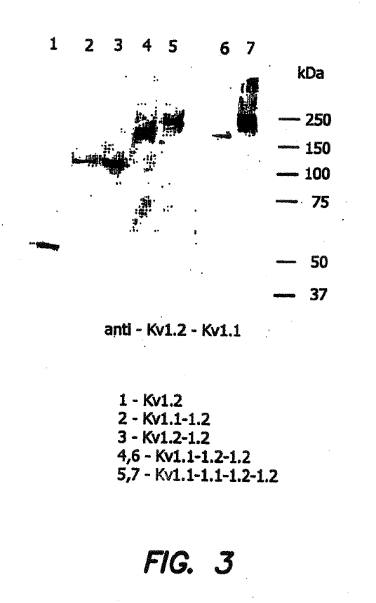

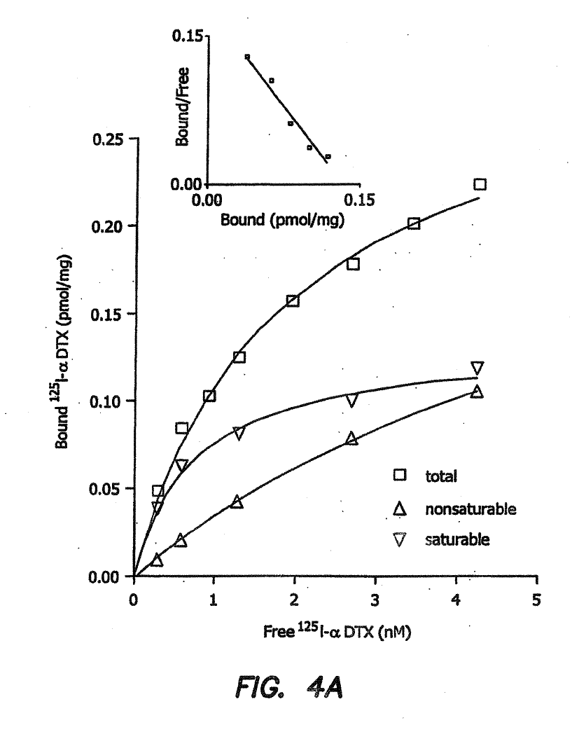

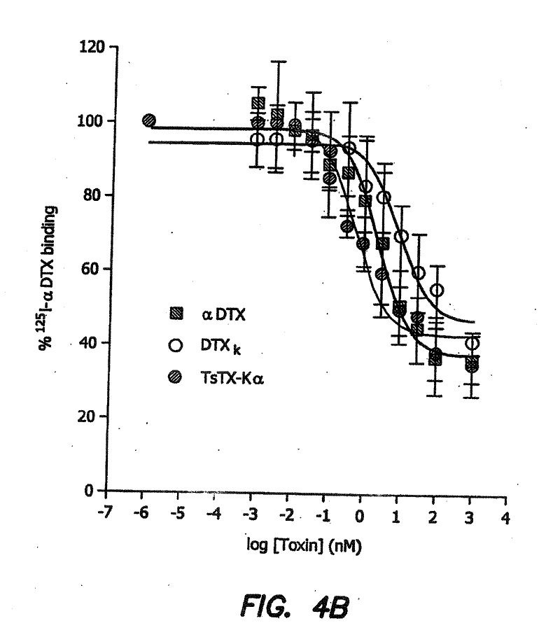

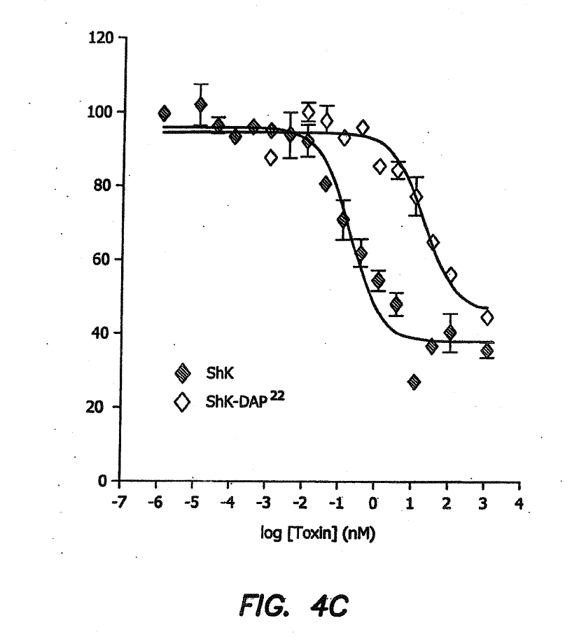

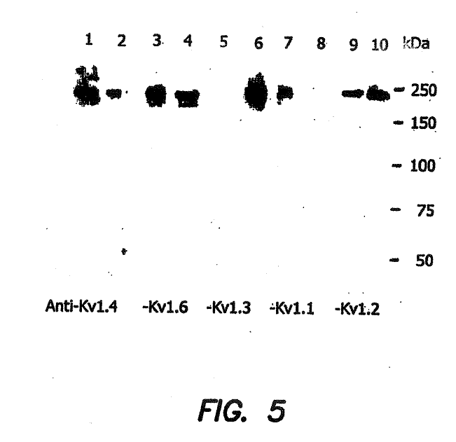

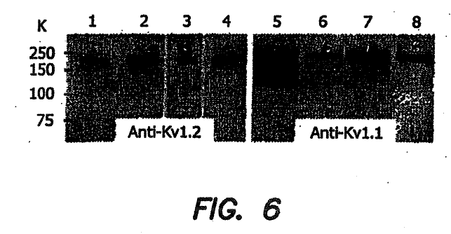

This kind of approach can be used generally for the preparation of any type of oligomeric proteins with desired pre-determined stoichiometry and composition. Proof of this versatile procedure is provided by the demonstrated successful expression in HEK 293 cells of Kv1.X channels containing three or four concatenated α subunits in different positional arrangements, as shown SDS-PAGE and Western blotting (FIG. 3). Channels containing Kv1.2, Kv1.1-1.2, Kv1.2-1.2 (FIG. 3), or any combination of subunits could be similarly obtained. (See e.g., FIG. 5). Importantly, these recombinant channels can be incorporated into the plasmalemma in fully-functional form; this was established from the saturable, high-affinity binding to the intact cells of a radiolabelled specific inhibitor of Kv1.X channels (FIG. 4), and confirmed by electrophysiological recording of voltage-activated K⁺ current.

Also, the functional properties of fully functional Kv1 recombinant channels Kv1.1-1.1-1.2-1.2, Kv1.1-1.2-1.1-1.2, Kv1.2-2.1-1.1-1.1, Kv1.2-1.1-1.2-1.1, Kv1.4-1.6-1.1-1.2 and Kv1.4-1.6-1.3-1.2 were examined by whole-cell, voltage-clamp, conventional recordings from HEK-293 cells transfected separately with each of the 6 constructs. The sensitivities of the potassium channel currents mediated by these tetramers to various channel blockers including tetraethylammonium (TEA), αDTX, DTXₖ, 4-aminopyridine (4AP), Agitoxinl (AgTX1), TsTX-Kα and ShK-Dap²² were determined. The K⁺ currents generated by these tetramers could be distinguished biophysically and by susceptibilities to inhibitors. The importance of α subunit arrangements within these tetramers in governing pharmacological differences and how oligomeric subtypes can be distinguished with appropriate blockers was established.

### Example 1

### Construction of Nucleic Acid Cassettes Encoding Kv1.1 and Kv1.2 Monomeric Subunits

Rat potassium channel Kv1.1 cDNA and Kv1.2 cDNA were provided separately in plasmid pAKS for use as PCR templates as described in Akhtar et al., J. Biol. Chem., 277:19, 16376-16382 (May 10, 2002). Similar techniques can be used for the cloning of human genes.

For the Kv1.1 subunit, the forward PCR primer has the nucleotide sequence:

(SEQ ID. NO: 118)
GTCTAGAATGACGGTGATGTCAGGGGAGAATGC,

wherein the underlined portion of SEQ ID NO: 118 indicates an Xba I restriction endonuclease site.

The reverse Kv1.1 PCR primer has the nucleotide sequence:

(SEQ ID NO: 119)
GCTCGAGAACATCGGTCAGGAGCTTGCTCTTATTAAC.

The underlined portion of SEQ ID NO: 119 indicates an Xba I restriction endonuclease site.

For the Kv1.2 subunit, the forward PCR primer has the nucleotide sequence

(SEQ ID. NO: 120)
GTCTAGAATGACAGTGGCTACCGGAGACCCAGTGG,

wherein the underlined portion of SEQ ID NO: 120 indicates an Xba I restriction endonuclease site.

The reverse Kv1.2 PCR primer has the nucleotide sequence:

(SEQ ID NO: 121)
GCTCGAGGACATCAGTTAACATTTTGGTAATATTCAC.

The underlined portion of SEQ ID NO: 121 indicates an Xba I restriction endonuclease site.

An equivalent version of this latter primer containing two in-frame (complementary or “antisense”) translational termination codons as well as the Xba I site has the nucleotide sequence:

(SEQ ID NO: 122)
GCTCGAGTTATCAGACATCAGTTAACATTTTGGTAATATTCAC

PCR amplification employing each of the Kv1.1 and Kv1.2 cDNAs were performed using standard methods and equipment as follows. The template DNA was denatured at 95° C. for 2 minutes, and amplification commenced using Taq polymerase in the presence of the indicated primer pairs for twenty two cycles of denaturation at 94° for 30 seconds, annealing at 58° C. for 45 seconds and elongation at 72° C. for 2 minutes.

PCR polymerization products were subjected to agarose gel electrophoresis. Gel slices were taken corresponding to bands having the expected molecular weight of nucleic acids encoding the full-length Kv subunit. These purified PCR products were cloned into PCR-blunt plasmid obtained from Invitrogen, Inc.; 5 μl purified PCR products containing approximately 100 ng DNA were mixed with 1 μl PCR-blunt plasmid and DNA ligase, and ligation was performed at 16° C. overnight.

The ligated DNA constructs were used to transform 50 μl aliquots of competent E. coli dh5a cells and positive clones selected on the basis of kanamycin resistance. Plasmid DNA was then isolated from larger cultures made from positive clones using a Midi-prep kit employing anion exchange column chromotography, purchased from Qiagen, Inc. and stored for future use.

In a similar fashion to that shown above for Kv1.1 and 1.2, Xho I and Xba I sites are introduced into each of the Kvα1.X remaining potassium channel subunits using PCR primer pairs. It is important that the number of nucleotides in the primer corresponding to the 3′ end of the insert be maintained so as to ensure that a spliced additional subunit, if any, be translated in frame.

For the preparation of subunit insert cassettes for the construction of expression vector encoding a multimeric protein, PCR-blunt DNA was digested simultaneously with Xho I and Xba I enzymes and excised inserts purified by agarose gel electrophoresis prior to use.

### Example 2

### Cloning of Kv1.X Inserts into Mutated pβUT2 Plasmid and Incorporation of 5′ and 3′ UTRs from Xenopus β-Globin

The multiple cloning site of plasmid pbUT2 was mutated as follows to destroy the Sal I, Bam H1 and Bgl II sites, thereby preventing interference with the MCS of pIRES2-EGFP (described in BD Biosceiences Clonetech Publication PR19951, published Oct. 3, 2002), which has similar sites.

In brief, pbUT2 plasmid was digested with Bgl II enzyme for 60 minutes at 37° C. The resulting 5′ Bgl II overhangs were filled using Klenow enzyme and dNTPs, and the resulting product was blunt-end ligated overnight using T4 ligase. BamH I and Sal I sites were destroyed in similar fashion in the DNA purified from positive clones.

Destruction of Sal I, Bam H1 and Bgl II sites in the resulting pbUT2 DNA was confirmed by restriction digestion of the triply-mutated DNA, with non-mutated pbUT2 serving as a control. Mutated pbUT2 was sequenced using bacteriophage T3- and T7-promoter specific primers, including the 5′- and 3′-untranslated regions (UTR) of Xenopus β-globin gene flanking the MCS in pbUT2. It has previously been shown that inclusion of these UTRs can enhance expression of potassium channel subunits in mammalian cells several-fold. See e.g., Linman et al., Neuron 9:861-871 (1992).

These nucleotide sequences were used to design primers for the second round of PCR. The above-noted purified Kv1.1- and 1.2-inserts (in Example 1) were ligated into mutated pbUT2 plasmid digested with Xba I and Xho I restriction endonucleases.

### Second Round of PCR-Amplification of Kv1.1- and 1.2 Genes.

The aim of the second round of PCR was to attach linker regions to each side of Kv1.X genes, with flanking restriction sites for their positional cloning into the master pIRES2-EGFP expression plasmid.

Forward and reverse primers used to incorporate Kv1.1- and 1.2-inserts were based on the nucleotide sequence of the 5′ and 3′ Xenopus b-globin untranslated regions (UTRs) of pbUT2 flanking the XbaI and XhoI sites of the MCS.

The annealing portions of the forward and reverse primers were:

| | |
|---|---|
| AGAATAAACGCTCAACTTTGGCAGATC,, | (SEQ ID NO: 123) |
| and | |
| CCAGATCCGGTACCAGATCGATCTCGAC. | (SEQ ID NO: 124) |

### Position 1 of the Tetramer

The unique Nhe I and Bgl II sites of the pIRES2-EGFP MCS were employed for insertion of the Kv1.X gene encoding the subunit occupying the first (N-terminal most) position of the four subunits comprising a Kv1.X tetramer, by incorporating the restriction endonuclease recognition sequence into the region flanking the annealing part of the UTR-specific forward and reverse primers, respectively. The Nhe I site is placed on the 5′ side of the sense primer, and the Bgl II site is placed on the 5′ side of the antisense primer.

### Position 2 of the Tetramer

Likewise, a Bgl II site (for incorporation on the 5′ side of the Kv1.x subunit gene) and an Eco RI site (for incorporation on the 3′ side of the Kv1.x subunit gene) is incorporated into the sense and antisense primers, respectively, for subunits engineered to occupy the second position of the tetrameric protein. Use of the corresponding sites of pIRES2-EGFP permit insertion of this cassette into the second position of the expression vector for making the tetrameric protein.

### Position 3 of the Tetramer

The third group of UTR-specific primers were flanked with Eco RI (forward “sense” primer) and Sal I sites (reverse “antisense” primer), respectively.

### Position 4 of the Tetramer

The fourth group of UTR-specific primers were flanked with Sal I (forward “sense” primer) and Bam H1 sites (reverse “antisense” primer), respectively. These sites provided the fourth (C terminal) subunit constituent of the tetrameric protein. For subunits intended to occupy this position, one or more translational stop codons are also incorporated into the 3′ end of the gene, as exemplified above.

In each case, PCR amplification was performed under the same conditions as described above for the first round of PCR amplification, with PCR products being purified by electrophoresis on agarose gel and cloned into PCR-blunt plasmid.

### Example 3

### Assembly of Kv1.X Tetramers of Specified Structure

In this Example, an expression vector for a specific tetramer having a Kv1.1 subunit in the first and second position, and a Kv1.2 subunit in the third and fourth position was made.

Tetrameric construct assembly began by incorporation of the Kv1.2 (+stop codon) insert for expression in the fourth position of tetramer. Kv.1.2 subunits containing a stop codon were excised from PCR-blunt DNA with Sal I and Bam HI enzymes, in parallel with digesting the pIRES2-EGFP plasmid with the same restrictases. These cleaved DNAs were then purified by electrophoresis on agarose gel. The purified vector and insert DNAs were then ligated together in the manner outlined above and used to transform DH5α cells. Positive transformant colonies of DH5α cells were used for DNA preparation of the plasmid containing the Kv1.2 (+stop) insert (termed Kv1.2 (#4)-pIRES-2-EGFP).

In the next step, the resultant Kv1.2 (#4)-pIRES-2-EGFP) DNA acted as the recipient for putting either a) the Kv1.2 (−stop codon) or b) Kv1.1 (−stop codon) inserts into the third position. To do this, the recipient plasmid DNA and the respective Kv1.1 or Kv1.2 PCR-blunt DNA were each digested with EcoRI and Sal I enzymes. Purification of the inserts and digested plasmid, ligation, DH5a cells transformation, and plasmid preparations were performed as before.

The same methodology used to prepare plasmid DNA with dimeric inserts spanning positions #3 and #4-Kv1.2 (#3)-1.2 (#4) and Kv1.1 (#3)-1.2 (#4) were used to clone Kv1.1(−stop)- or Kv1.2(−stop)-inserts respectively, into position #2 using Bgl II and Eco RI cloning sites.

Finally, completion of the tetramer assembly entailed insertion as before of Kv1.1 (−stop) into position #1 by digestion of insert and recipient plasmids with Nhe I+Bgl II. Recipient plasmids used for this stage of the construction were: Kv1.1(#2)-1.2(#3)-1.2(#4) and Kv1.2(#2)-1.1(#3)-1.2(#4).

FIG. 1 shows a schematic representation of the construction of Kv1.1(#1)-Kv1.1(#2)-Kv1.2(#3)-KV1.2(+S)(#4) pIRES2-EGFP.

FIG. 2A shows digestion and agarose gel electrophresis of intermediate Kv1.1 constructs to verify the assembly of the subunit monomers in the desired position of the expression vector. Thus Lane 1 of this figure shows Kv1.2(#4)-pIRES2-EGFP digested with Sal I and Bam H1.

Lane 2 shows digestion of Kv1.2(#3)-Kv1.2(#4)-pIRES2-EGFP with Sal I and Eco RI to remove only the insert in the third position. Lane 3 shows digestion of Kv1.2(#3)-Kv1.2(#4)-pIRES2-EGFP with Eco RI and Bam HI to remove a single dimeric insert.

Lane 4 shows digestion of Kv1.1(#2)-Kv1.2(#3)-Kv1.2(#4)-pIRES2-EGFP with Bgl II and Eco RI to remove the Kv1.1-encoding insert. Lane 5 shows digestion of Kv1.1(#2)-Kv1.2(#3)-Kv1.2(#4)-pIRES2-EGFP with Bgl II and Bam H1 to remove the trimeric Kv1.1-Kv1.2(#3)-Kv1.2(#4) insert.

Lane 6 shows digestion of Kv1.1(#1)-Kv1.1(#2)-Kv1.2(#3)-Kv1.2(#4)-pIRES2-EGFP with Bgl II and Nhe I to remove the Kv1.1(#1)-encoding insert. Lane 7 shows digestion of Kv1.1(#1)-Kv1.1(#2)-Kv1.2(#3)-Kv1.2(#4)-pIRES2-EGFP with Bam H1 and Nhe I to remove the entire Kv1.1(#1)-Kv1.1(#2)-Kv1.2(#3)-Kv1.2(#4) tetramer-encoding insert.

In FIG. 2B, agarose gel electrophoresis is performed on Kv1.1(#1)-Kv1.1(#2)-Kv1.2(#3)-Kv1.2(#4)-pIRES2-EGFP plasmid; Lane 1 and 6 indicate linearization of the plamsid using restriction endonuclease Nhe 1; Lane 2 shows the result of a digestion with Nhe I and Bgl II to liberate a Kv 1.1 momomer. Lane 3 shows the result of digestion with Nhe 1 and Eco RI to liberate a Kv1.1-Kv1.1 dimer. Lane 4 shows the result of digestion with Nhe 1 and Sal 1 to liberate a Kv1.1-Kv1.1-Kv1.2 trimer. Lane 5 shows the result of digestion with Nhe I and Bam HI to liberate the entire tetramer.

### Example 4

### Expression of Kv1.X Constructs

Individual cultures of HEK 293 cells were transfected with a) Kv1.2(#4)-pIRES2-EGFP, b) Kv1.2(#3)-Kv1.2(#4)-pIRES2-EGFP, c) Kv1.1(#3)-Kv1.2(#4)-pIRES2-EGFP, d) Kv1.1(#2)-Kv1.2(#3)-Kv1.2(#4)-pIRES2-EGFP, and e) Kv1.1(#1)-Kv1.1(#2)-Kv1.2(#3)-Kv1.2(#4)-pIRES2-EGFP and permitted to incubate for 48 hours at 37° C.

Cells from each culture were then prepared by boiling for 3 minutes in 0.5% SDS PAGE reducing buffer, and electrophoresis in a polyacrylamide gel. Following completion of the electrophoresis, the gel was used for electrotransfer of separated proteins onto a nitrocellulose membrane. The membrane was incubated with a primary anti-Kv1.1 or anti-Kv1.2 antibody. A secondary labelled antibody was used to detect the presence of a complex between the first antibody and the Kv1.x protein on the nitrocellulose membrane. For detection, a horse-radish peroxidase-conjugated secondary antibody was used; signals were visualised using an ECL kit (GE). The membrane was then developed in a standard detection step.

FIG. 3 shows the results of this experiment. Lane 1 shows expression of Kv1.2 momomer from cultures transfected with Kv1.2(#4)-pIRES2-EGFP. Lane 2 shows expression of Kv1.1-Kv1.2 dimer from cultures transfected with Kv1.1(#3)-Kv1.2(#4)-pIRES2-EGFP. Lane 3 shows expression of Kv1.2-Kv1.2 from cultures transfected with Kv1.2(#3)-Kv1.2(#4)-pIRES2-EGFP. Lane 4 shows expression of Kv1.1-Kv1.2-Kv1.2 from cultures transfected with Kv1.1(#2)-Kv1.2(#3)-Kv1.2(#4)-pIRES2-EGFP. Lane 5 shows expression of Kv1.1-Kv1.1-Kv1.2-Kv1.2 from cultures transfected with Kv1.1(#1)-Kv1.1(#2)-Kv1.2(#3)-Kv1.2(#4)-pIRES2-EGFP. All these Western blots were detected using anti-Kv1.2 antibody.

Lanes 6 and 7 are identical to Lanes 5 and 6, except the Western blots were detected using anti-Kv1.1 antibody rather than anti-Kv1.2 antibody.

### Example 5

### Expression of Functional K⁺ Channels

HEK 293 cells are transiently transfected with a PIRES2-EGFP vector encoding Kv1.1-1.1-1.2-1.2 (also referred to as forward adjacent channel; see below) and the cells incubated to display the potassium channels on the cell membranes.

Cells are harvested in 25 mM Tris HCl, 5 mM KCl pH 7.5 Saturatable binding of ¹²⁵I-α-dendrotoxin (DTX), a Kv1-selective toxin, was performed using a filtration assay to measure the amount of assembled channel targeted to the plasmalemma. Transfected cells suspended in binding buffer were incubated for 1 hour with increasing concentrations of labelled DTX; for non-saturable binding experiments, the incubation is performed in the presence of 1 μM unlabeled DTX. Measurements are made in triplicate under established conditions by rapid filtration through GF/F glass microfiber filters presoaked with 0.5% (w/v) polyethyleneimine. The radioactivity associated with the washed filters was quantified by γ-radiation counting.

FIG. 4A shows the selective binding curves of ¹²⁵I-αDTX for this tetramer, wherein the inverted triangle shows saturable binding, the triangle shows the curve for non-saturable binding, while the square shows the curve for total binding.

FIG. 4B shows competition experiments of 2.5 nM ¹²⁵I-αD1× to transfected HEK-293 cells by αDTX (closed circle), DTXₖ (open circle), or TsTx-Kα (tityustoxin-Kα) (square), in which ¹²⁵I-αDTX is readily displaced from the Kv1.1-1.1-1.2-1.2 tetramer by these toxins.

In FIG. 4C a competition experiment is performed incubating transfected HEK cells with 2.5 μM ¹²⁵I-αDTX in the presence of various concentrations of ShK and its derivative ShK-Dap²². Similarly, Shk antagonizes ¹²⁵I-αDTX binding to a greater extent than does the derivative ShK-Dap²².

### Example 6

### Construction of Nucleic Acid Cassettes Encoding Rat Kv1.3, Kv1.4 and Kv1.6 Monomeric Subunits

Potassium channel Kv1.3 cDNA, Kv1.4 cDNA and Kv1.6 cDNA pAKS plasmids served as PCR templates.

For the Kv1.3 subunit, the forward PCR primer has the nucleotide sequence:

(SEQ ID. NO: 127)
GTCTAGAATGACCGTGGTGCCCGGGGACCACCTG,

wherein
the underlined portion of SEQ ID NO: 127 indicates an Xba I restriction endonuclease site.

The reverse Kv1.3 PCR primer has the nucleotide sequence:

(SEQ ID NO: 128)
GCTCGAGGACATCAGTGAATATCTTTTTGATGTTGACAC.

### The

underlined portion of SEQ ID NO: 128 indicates an Xba I restriction endonuclease site.

For the Kv1.4 subunit, the forward PCR primer has the nucleotide sequence

(SEQ ID. NO: 129)
GTCTAGAATGGAGGTGGCAATGGTGAGTGCC,

wherein the underlined portion of SEQ ID NO: 129 indicates an Xba I restriction endonuclease site.

The reverse Kv1.4 PCR primer has the nucleotide sequence:

(SEQ ID NO: 130)
GCTCGAGGACACATCAGTCTCCACAGCCTTTGCATTAG.

The underlined portion of SEQ ID NO: 130 indicates an Xba I restriction endonuclease site.

For the Kv1.6 subunit, the forward PCR primer has the nucleotide sequence

(SEQ ID. NO: 131)
GTCTAGAATGAGATCGGAGAAATCCCTTACGC,

wherein the underlined portion of SEQ ID NO: 131 indicates an Xba I restriction endonuclease site.

The reverse Kv1.6 PCR primer has the nucleotide sequence:

(SEQ ID NO: 132)
GCTCGAGGAGACCTCCGTGAGCATTCTTTTCTCTGC.

The underlined portion of SEQ ID NO: 132 indicates an Xba I restriction endonuclease site.

PCR amplification employing each of the Kv1.3, Kv1.4 and Kv1.6 cDNAs were performed using standard methods and equipment as described in Example 1.

In a similar fashion to that shown above for Kv1.1 and 1.2 in Example I, Xho I and Xba I sites were introduced into each of these potassium channel subunits using specially designed PCR primer pairs. It is important that the number of nucleotides in the primer corresponding to the 3′ end of the insert be maintained so as to ensure that a spliced additional subunit, if any, be translated in frame.

For the preparation of cassettes for the construction of expression vector encoding a multimeric protein, PCR-blunt DNA was digested simultaneously with Xho I and Xba I enzymes and excised inserts purified by agarose gel electrophoresis prior to use.

### Example 7-11

### Assembly of Kv1.X Tetramers of Specified Structure

In these Examples, expression vectors for five specific concatameric tetramers having the following subunits were made essentially as described in Example 3:

1. a Kv1.2 subunit in the first and second position, and a Kv1.1 subunit in the third and fourth position (also referred to as reverse adjacent channel);
2. a Kv1.1 subunit in the first and third position, and a Kv1.2 subunit in the second and fourth position (also referred to as forward diagonal channel);
3. a Kv1.2 subunit in the first and third position, and a Kv1.1 subunit in the second and fourth position (also referred to as reverse diagonal channel);
4. a Kv1.4 subunit in the first position, a Kv1.6 subunit in the second position, a Kv1.1 subunit in the third position and a Kv1.2 subunit in the fourth position; and
5. a Kv1.4 subunit in the first position, a Kv1.6 subunit in the second position, a Kv1.3 subunit in the third position and a Kv1.2 subunit in the fourth position.

It will be understood by those of skill in the art, the methods and compositions of the present invention are equally applicable to any combination, order or stoichiometry of subunits in a multimeric protein. The drug screening aspects of the present invention are thus equally applicable to ion channel subunits, Na+ channel subunits, Ca++ channel subunits, K+ channel subunits, Kv1.x subunits, and the like.

### Example 12-13

### Expression of Kv1.X Constructs

Individual cultures of HEK 293 cells were transfected with each of a) Kv1.4(#1)-Kv1.6(#2)-Kv1.1(#3)-Kv1.2(#4)-pIRES2-DsRed and b) Kv1.4(#1)-Kv1.6(#2)-Kv1.3(#3)-Kv1.2(#4)-pIRES2-DsRed and permitted to incubate for 48 hours at 37° C.

Direct immunoblotting of lysates from the cells above was performed as described in Example 4. FIG. 5 shows immunoblots of lysates of cells transfected with tetrameric constructs in pIRES-DsRed. The lysates were solubilized in LDS sample buffer and immunoprobed with antibodies specific for each of the constituent subunits, Kv1.1, Kv1.2, Kv1.3, Kv1.4 and Kv1.6. The immunoblot results for the tetramer Kv1.4-1.6-1.1-1.2 is shown in lanes 1, 3, 5, 7, 9. The immunoblot results for the tetramer Kv1.4-1.6-1.3-1.2 is shown in lanes 2, 4, 6, 8, 10. Lysates prepared from untransfected cells showed no specific immunoreactivity against the antibodies used.

### Example 14-17

### Expression of Kv1.X Constructs

Individual cultures of HEK 293 cells were transfected with each of: Kv1.1(#1)-Kv1.1(#2)-Kv1.2(#3)-Kv1.2(#4)-pIRES2-EGFP; Kv1.1(#1)-Kv1.2(#2)-Kv1.1(#3)-Kv1.2(#4)-pIRES2-EGFP; Kv1.2(#1)-Kv1.2(#2)-Kv1.1(#3)-Kv1.1(#4)-pIRES2-EGFP; and Kv1.2(#1)-Kv1.1(#2)-Kv1.2(#3)-Kv1.1(#4)-pIRES2-EGFP. The cultures were permitted to incubate for 48 hours at 37° C. The Kv1.1-1.1-1.2-1.2 tetramer is also referred to as a forward adjacent channel), Kv1.1-1.2-1.1-1.2 is also referred to as a forward diagonal channel), Kv1.2-1.2-1.1-1.1 is also referred to as a reverse adjacent channel), and Kv1.1.2-1.1-1.2-1.1 is also referred to as a reverse diagonal channel, and the resulting transfected cells were allowed to display the potassium channels on the cell membranes.

The functional potassium channels on the surface were biochemically analyzed. This was accomplished by biotinylation of the surface components of HEK-293 cells transfected with the individual tandem-linked constructs.

For surface biotinylation, HEK-293 cells transfected as above for 48 hours with these concatenated genes in pIRES2-EGFP were harvested, washed, resuspended at about 2-3×10⁷ cells/ml of PBS, and incubated with 1 mg/ml sulfo-NHS-LC-biotin (Pierce) at room temperature for 30 min. Remaining reagent was quenched with 100 mM glycine for 30 min, samples were solubilised in 2% Triton X-100 for 1 h at 4° C., and centrifuged at 100 000 g for 1 h. The supernatants were incubated with streptavidin-agarose (Pierce) (70 l slurry/ml) overnight at 4° C. with rotation. After washing the pelleted streptavidin-agarose with ice-cold TBS containing 0.1% Tween-20, bound proteins were dissolved in SDS-PAGE sample buffer before Western blotting.

The solubilised biotinylated proteins were precipitated with streptavidin agarose. FIG. 6 shows a Western blot with anti-Kv1.1 and 1.2 IgGs where, for each of the 4 oligomers, intact tetrameric channels containing both subunit types were expressed on the surface. These tetramers had an apparent molecular weight of ˜240 kDa; an intact protein band (Mr ˜240 k) was obtained from all 4 constructs, indicative of the successful expression of intact heterotetramers.

FIG. 7 shows the results of restriction mapping digests of tetrameric constructs and intermediates. FIG. 7A shows the sequential release from plRES2-EGFP Kv1.1-1.1-1.2-1.2 of Kv1.1, Kv1.1-1.1, Kv1.1-1.1-1.2 or Kv1.1-1.1-1.2-1.2 inserts, as seen in lanes 1-4 from final Kv1.1-1.1-1.2-1.2 and Kv1.1, Kv1.1-1.2, Kv1.1-1.2-1.1 or Kv1.1-1.2-1.1-1.2 inserts from final pIRES2-EGFP Kv1.1-1.2-1.1-1.2 by digestion with Nhe I and Bgl II, Eco Rl, Sal I or Bam HI, as seen in lanes 5-8 from an agarose (1%) gel electrophoretogram. FIG. 7B shows the sequential release of Kv1.2, Kv1.2-1.2, Kv1.2-1.2-1.1 or Kv1.2-1.2-1.1-1.1 inserts from the final plRES2-EGFP Kv1.2-1.2-1.1-1.1, and, Kv1.2, Kv1.2-1.1, Kv1.2-1.1-1.2 or Kv1.2-1.1-1.2-1.1 inserts from the final pIRES2-EGFP Kv1.2-1.1-1.2-1.1 by digestion with Nhe I and Bgl II, EcoR I, Sal I or Bam HI, as seen in lanes 9-12 and 13, 14, 15 and 17 respectively, and lane 16 represents un-digested or complete pIRES2-EGFP Kv1.2-1.1-1.2-1.1 from an agarose (1%) gel electrophoretogram. The letter “M” in FIG. 7A and FIG. 7B indicates the apparent molecular weight in kbp.

### Example 18-20

### Surface Expression of K Channels

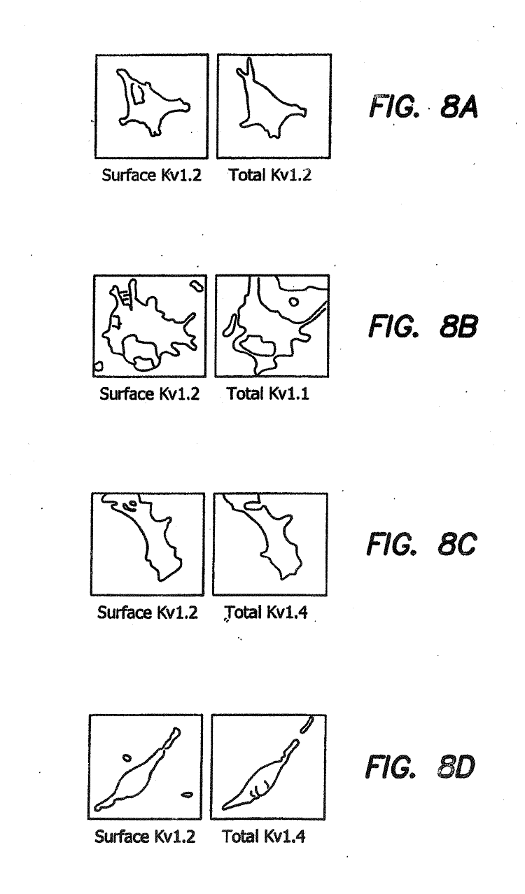

In these examples, expression of the potassium channels was visualized by double immuno-staining and fluorescence microscopy of COS-7 cells transfected with each of the constructs encoding the tetramers Kv1.1-1.1-1.2-1.2, Kv1.4-1.6-1.1-1.2 and Kv1.4-1.6-1.3-1.2. The COS-7 cells expressing these constructs were labelled with IgGs specific for external epitopes of Kv1.2, and followed by fluorescent microscopy. Fluorescence microscopy of COS-7 cells transfected with the constructs encoding representative heterotetramers using Lipofectamine 2000 was performed according to a surface staining protocol (Tiffany et al., J. Cell. Biol. 148:147-158, (2000). FIGS. 8A, 8B, 8C and 8D show fluorescent micrographs indicating surface expression of 3 fully functional hetero-tetrameric Kv1 channels in COS-cells transfected with the corresponding constructs encoding the tetramers. COS-7 cells, transfected with Kv1.1-1.1-1.2-1.2-pIRES2-EGFP (expression of the corresponding functional channel seen in FIGS. 8A and 8B), Kv1.4-1.6-1.1-1.2-plRES2-EGFP (the expression of the corresponding functional channel seen in FIG. 8C) or Kv1.4-1.6-1.3-1.2-pIRES2-DsRed (the expression of the corresponding functional channel seen in FIG. 8D) were double labelled using an antibody reactive with external epitopes of Kv1.2 (left panels) and monoclonal antibodies specific for Kv1.2 (FIG. 8A), Kv1.1 (FIG. 8B) or Kv1.4 (FIGS. 8C and 8D) (right panels). In all FIGS. 8A-8D, surface labeling was observed (left panels); the surface labelling was distinguished from the total labeling (right panels) using anti-species IgGs coupled to Alexa Fluor 594 or 488, respectively. Only background signals were seen upon omission of primary antibodies.

Similarly, cell surface expression was demonstrated for the tetramers Kv1.1-1.2-1.1-1.2, Kv1.2-1.2-1.1-1.1 and Kv1.2-1.1-1.2-1.1.

From these examples, it is established that the above concatenated heteromers successfully traffic intact to the plasma membrane in active form when expressed in mammalian cells. The examples also validate the utilisation of recombinant Kv1 channels as targets for future drug screening.

### Example 21-23

### Comparison of Functional Properties of Heteromeric Tetramer Constructs

In these examples, the functional properties of the concatenated Kv1 heteromers Kv 1.1-1.1-1.2-1.2, Kv1.4-1.6-1.1-1.2 and Kv1.4-1.6-1.3-1.2 were examined by whole-cell voltage-clamped recordings from the corresponding transfected HEK-293 cells. The activation kinetics of the currents mediated by Kv 1.1-1.1-1.2-1.2 Kv 1.4-1.6-1.1-1.2 and Kv1.4-1.6-1.3-1.2 were determined.

The sensitivities of the potassium channel currents mediated by Kv 1.1-1.1-1.2-1.2 Kv 1.4-1.6-1.1-1.2 and Kv1.4-1.6-1.3-1.2 to various blockers were also examined in these examples.

Whole-cell voltage-clamp experiments were performed as outlined in Sokolov, et al., Neuropharmacol. 53:2, 272-282, (2007), whose disclosure is hereby incorporated by reference. The internal solution was (in mM): 90 KCl, 50 KF, 30 KOH, 10 EGTA, 20 HEPES, pH 7.4. The extracellular bath solution contained (in mM) 135 choline chloride, 20 KOH, 1.8 CaCl₂, 1 MgCl₂, and 40 HEPES, pH 7.4, and 0.01% (w/v) BSA. Solutions were exchanged by continuous flow with a peristaltic pump or a Cellectricon Dynaflow-16 system. Series resistance compensation was applied to minimize the voltage error (<10 mV). Correction was made for a calculated liquid junction potential of +8.4 mV. Holding potential was −100 mV. Leak subtraction was used to isolate K⁺ current (I_{K}). Analogue traces were filtered at 5 kHz, and sampled at 50 kHz. Non-linear fitting was carried out with equations described in (Sack et al., Gen. Physiol. 128:1, 119-132, (2006), whose disclosure is hereby incorporated by reference. Data are reported as mean±standard error; n values refer to number of individual cells tested. Statistical significance was evaluated by two-tailed Student's t-test using data obtained from at least five independent experiments performed in parallel.

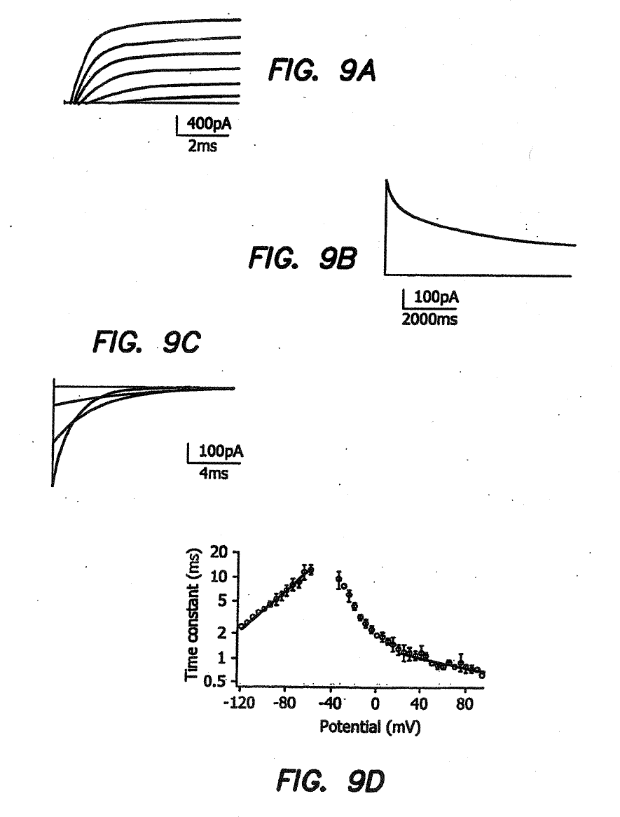

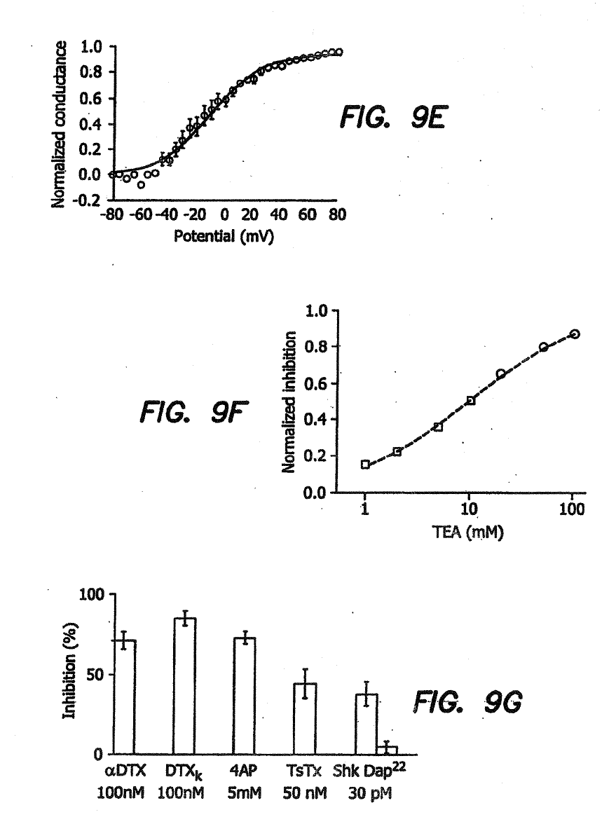

FIGS. 9A-9E show that the Kv1.1-1.1-1.2-1.2 construct expresses functionally uniform channels. FIG. 9A shows K⁺ currents (I_{K}) in Kv1.1-1.1-1.2-1.2 channels generated in response to depolarising steps (gray traces) from −40 to 80 mV in 20 mV increments are consistent with an exponential function (black lines). FIG. 9B shows inactivation of I_{K} in Kv1.1-1.1-1.2-1.2 channels during a pulse to 0 mV (gray traces), consistent with an exponential function (black line). FIG. 9C shows deactivation of I_{K} in Kv1.1-1.1-1.2-1.2 channels at −80, −100 or −120 mV (gray traces) after 50 ms at +60 mV, consistent with mono-exponential functions (black lines). FIG. 9D shows time constants (n=3) associated with activation (>−50 mV) or deactivation (<−50 mV) in Kv1.1-1.1-1.2-1.2 channels; lines consistent with exponential functions. FIG. 9E shows the conductance-voltage relationship of outward K⁺ peak currents (n=15) after 100 ms at indicated voltage in Kv1.1-1.1-1.2-1.2 channels; the dashed line is a Boltzmann fit.

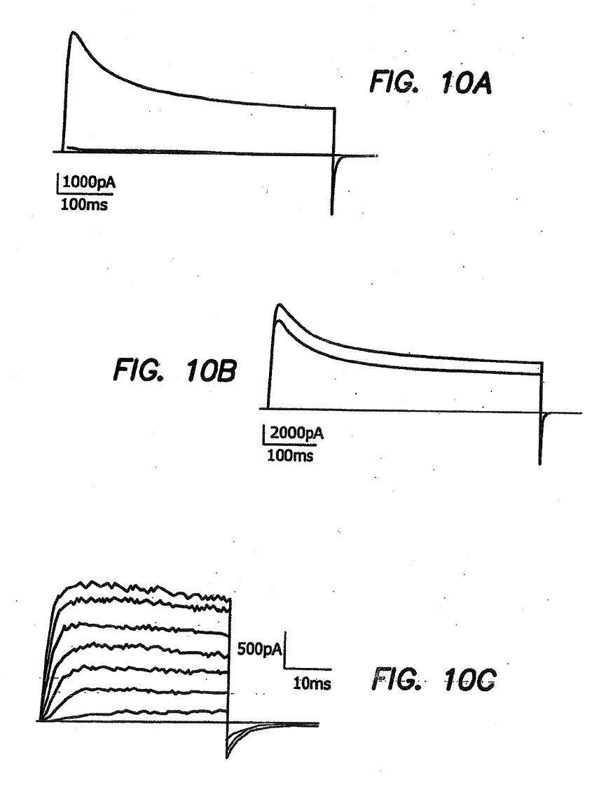

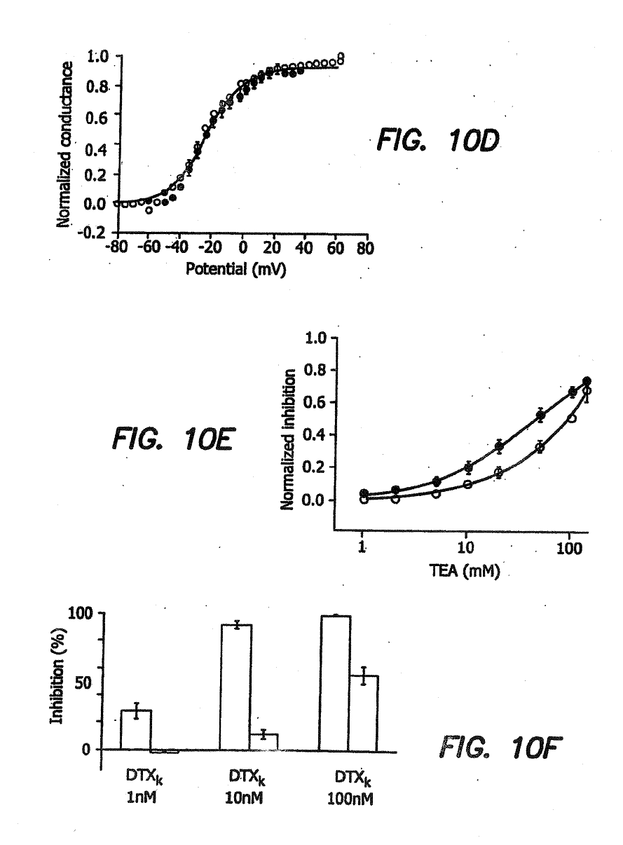

FIG. 10A-10E show that similar Kv1.4-containing hetero-tetrameric channels are distinguishable by DTXₖ and TEA intoxication (tetraethyl ammonium; more details regarding the inhibition given below). FIGS. 10A and 10B show the I_{K} from Kv1.4-1.6-1.1-1.2 and Kv1.4-1.6-1.3-1.2, respectively, induced by 500 ms voltage steps from −100 to 0 mV (black). The effect of 10 nM DTXₖ on I_{K} from Kv1.4-1.6-1.1-1.2 and Kv1.4-1.6-1.3-1.2 induced by 500 ms voltage steps from −100 to 0 mV is in gray. FIG. 10C shows representative current traces of Kv1.4-1.6-1.1-1.2 in response to depolarising steps from −100 to +80 mV in 20 mV increments. FIG. 10D shows gₖ-V relations assembled from peak outward currents from Kv1.4-1.6-1.1-1.2 (closed circles) and Kv1.4-1.6-1.3-1.2 (open circles).

The biophysical and pharmacological properties of the three channels Kv 1.1-1.1-1.2-1.2, Kv1.4-1.6-1.1-1.2 and Kv1.4-1.6-1.3-1.2 are tabulated in Table 1.

TABLE 1
Biophysical and pharmacological properties of
three hetero-tetrameric K⁺ channels

| | Kv1.1-1.1- | Kv1.4-1.6 | Kv1.4-1.6 |
|---|---|---|---|
| | 1.2-1.2 | 1.1-1.2 | 1.3-1.2 |
| τ-Activation at 0 mV | 1.9 ± 0.1 | 1.5 ± 0.1 | 1.8 ± 0.1 |
| (ms) | (n = 3) | (n = 7) | (n = 5) |
| τ₁-Inactivation at 0 | 540 ± 20 | 45 ± 8 | 47 ± 0.1 |
| mV (ms) | (n = 5) | (n = 7) | (n = 5) |
| τ₂-Inactivation at 0 | 6200 ± 500 | 260 ± 20 | 270 ± 6 |
| mV (ms) | (n = 5) | (n = 7) | (n = 5) |
| V½ (mV) | −12 ± 1.8 | −25 ± 0.6 | −23 ± 0.8 |
| | (n = 13) | (n = 10) | (n = 10) |
| Slope (k) (mV) | 18 ± 1.6 | 11 ± 0.7 | 10 ± 0.5 |
| | (n = 13) | (n = 10) | (n = 10) |

τ measures time following activation at which the channels are opened;
τ₁ and τ₂ are distinct time constants reflecting two inactivation states for the channels under study (values for activation and inactivation were calculated by fitting power and double-exponential functions, respectively.)
V½ is the voltage at which half of the channels are activated.
Slope k = conductance k/voltage mV.
Values for V½ and slope k were calculated from Boltzmann equation fitting of the gₖ-V relations from peak outward currents.

As mentioned earlier, sensitivities of the potassium channel currents mediated by Kv1.1-1.1-1.2-1.2, Kv1.4-1.6-1.1-1.2 and Kv1.4-1.6-1.3-1.2 to various blockers were also examined in these examples for each of these channels. Inhibition by tetraethylammonium (TEA) αDTX, DTXₖ, 4-aminopyridine (4AP), TsTX and ShK-Dap²² were determined.

FIG. 9F shows the concentration dependence of TEA inhibition of I_{K} (n=4) during voltage steps to +60 mV for 50 ms in Kv1.1-1.1-1.2-1.2 channels. Inhibition in each cell was normalized (open circles) by saturating value of a fit with Langmuir isotherm (dashed line).

FIG. 9G shows reduction by αDTX, DTXₖ, 4-aminopyridine (4AP), TsTX and ShK-Dap²² of I_{K} from Kv1.1-1.1-1.2-1.2 (open bars), homomeric Kv1.1 (gray bar) or Kv1.2 (black bar) during voltage steps to +60 mV for 50 ms (n≧5). ShK-Dap²² measurements were conducted in a solution used previously and described in Sokolov et al., Neuropharmacol. 53:2, 272-282, (2007), whose disclosure is hereby incorporated by reference.

FIG. 10E shows the concentration dependence for TEA inhibition of I_{K} from Kv1.4-1.6-1.1-1.2 (closed circles) or Kv1.4-1.6-1.3-1.2 (open circles) assayed by pulses to 0 mV (n=5) and fit by the Langmuir equation (dashed line) as in FIG. 10C.

FIG. 10F shows inhibition by 3 DTXₖ concentrations of I_{K} from Kv1.4-1.6-1.1-1.2 (open bars) or Kv1.4-1.6-1.3-1.2 (gray bars), assayed at ≦0 mV showed a significant sensitivity difference (1 nM, p=0.00029 (***); 10 nM, p=0.0002 (***); 100 nM, p=0.00034) (***).

The data from these examples indicate that the three tetramers are expressed as essentially uniform populations of Kv1 channels that can be distinguished biophysically and by susceptibilities to inhibitors. The K⁺ currents of the three tetramers can be distinguished by inactivation rates and/or by selective inhibitors.

### Example 24-27

### Comparison of Functional Properties of Heteromeric Tetramer Constructs

In these examples, the functional properties of the Kv1 heteromers Kv1.1-1.1-1.2-1.2, Kv1.1-1.2-1.1-1.2, Kv1.2-2.1-1.1-1.1 and Kv1.2-1.1-1.2-1.1 were compared by whole-cell, voltage-clamp, conventional recordings from HEK-293 cells transfected separately with each of the 4 constructs.

In these examples the sensitivities of the potassium channel currents mediated by these tetramers to various blockers were also examined. Inhibition by TEA, or Agitoxinl (AgTX1) and TsTX-Kα were determined.

Whole-cell voltage-clamp was performed as described in Example 25-27, except where specified. For larger scale recordings, an automated patch-clamp system (QPatch 16, Sophion, Denmark) was used; its disposable Qplates contain 16 individual and parallel patch-clamp positions. Cells were detached from culture plates with 0.05% trypsin/EDTA solution and kept in serum-free medium (CHO-S-SFM II [Invitrogen], 25 mM HEPES, pH 7.4, 0.04 μg/ml soya bean trypsin inhibitor, and 10 μg/ml penicillin-streptomycin) in an on-board stirred reservoir. Prior to testing, the cells were automatically transferred to an integral mini-centrifuge, pelleted, re-suspended in external solution (as detailed above) and washed before being applied to the pipeting wells in the Q plate. Giga-seals were formed upon execution of a combined suction/voltage protocol; gradually increasing suction leads to the whole-cell configuration. Compounds were applied, via a 4-way pipeting robot, through integrated glass-coated microfluidic flow channels. Liquid flow is laminar with exchange time constants in the range of 50-100 ms. After application, all fluids were collected in a built-in waste reservoir (250 l). Whole cell currents were measured at a holding potential of −100 mV, then depolarised to +60 mV for 200 ms or stepped from the holding potential in +10 mV increments. Data analysis was performed using an integrated database (Oracle) within Q Patch software (Sophion, Denmark).

For comparison, values for the biophysical parameters of all 4 heteromers are displayed in Table 2. The four concatamers showed only minor dissimilarities in terms of the parameters for activation, deactivation and gK-V relationship.

TABLE 2
Biophysical and pharmacological properties of
four concatameric K⁺ channels compared to a Kv1.1-1.2
dimer-containing channel

| | | | | Reverse | |
|---|---|---|---|---|---|
| | Adjacent | Reverse adjacent | Diagonal | diagonal | Dimer |
| A/τ at 0 mV, | 1.9 ± 0.1 | 2.3 ± 0.6 | 1.4 ± 0.1 | 1.3 ± 0.1 | 10.6 ± 1.3 |
| (ms) | (3) | (3) | (3) | (4) | (6) |
| fI/τ1 at | 540 ± 2 | 529 ± 50 | 506 ± 34 | 341 ± 17 | — |
| 0 mV (ms) | (5) | (4) | (3) | (4) | |
| sI/τ2 at | 6200 ± 500 | 5053 ± 596 | 3586 ± 485 | 4372 ± 404 | — |
| 0 mV, | (5) | (4) | (3) | (4) | |
| (ms) | | | | | |
| V½ (mV) | −13 ± 1 | −15 ± 1 | −29 ± 3 | −25 ± 1 | 0.7 ± 1.6 |
| | (18) | (6) | (11) | (15) | (9) |
| Slope | 22 ± 1 | 22 ± 2 | 24 ± 2 | 20 ± 2 | 17 ± 1.0 |
| (k) (mV) | (18) | (6) | (11) | (15) | (9) |
| IC₅₀ TEA | 9.7 ± 1 | 6.7 ± 2 | 0.76 ± 0.1 | 0.96 ± 0.14 | 12 ± 1 |
| (mM) | (9) | (4) | (5) | (5) | (8) |
| IC₅₀ | 5 ± 0.4 | 5 ± 0.5 | >1 μM | 1 μM | 39 ± 4 |
| AgTx1 | (23) | (13) | (4) | (5) | (13) |
| (nM) | | | | | |
| IC₅₀ | 26 ± 3 | 25 ± 7 | >1 μM | >1 μM | 33 ± 3 |
| TsTxKα | (3) | (3) | (4) | (3) | (3) |
| (nM) | | | | | |

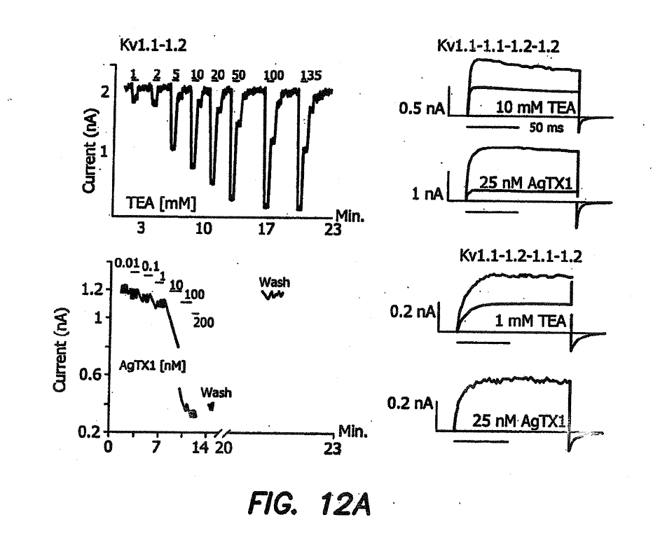

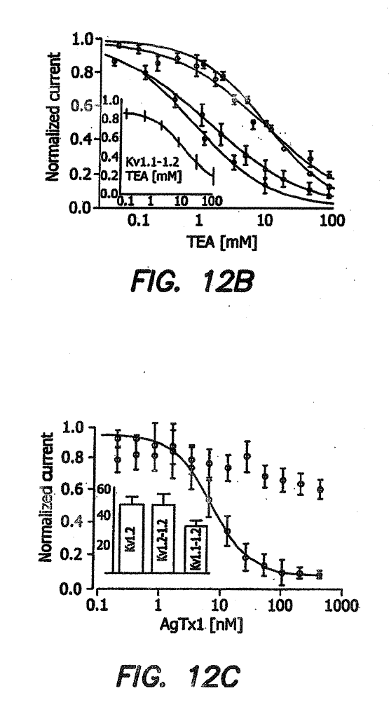

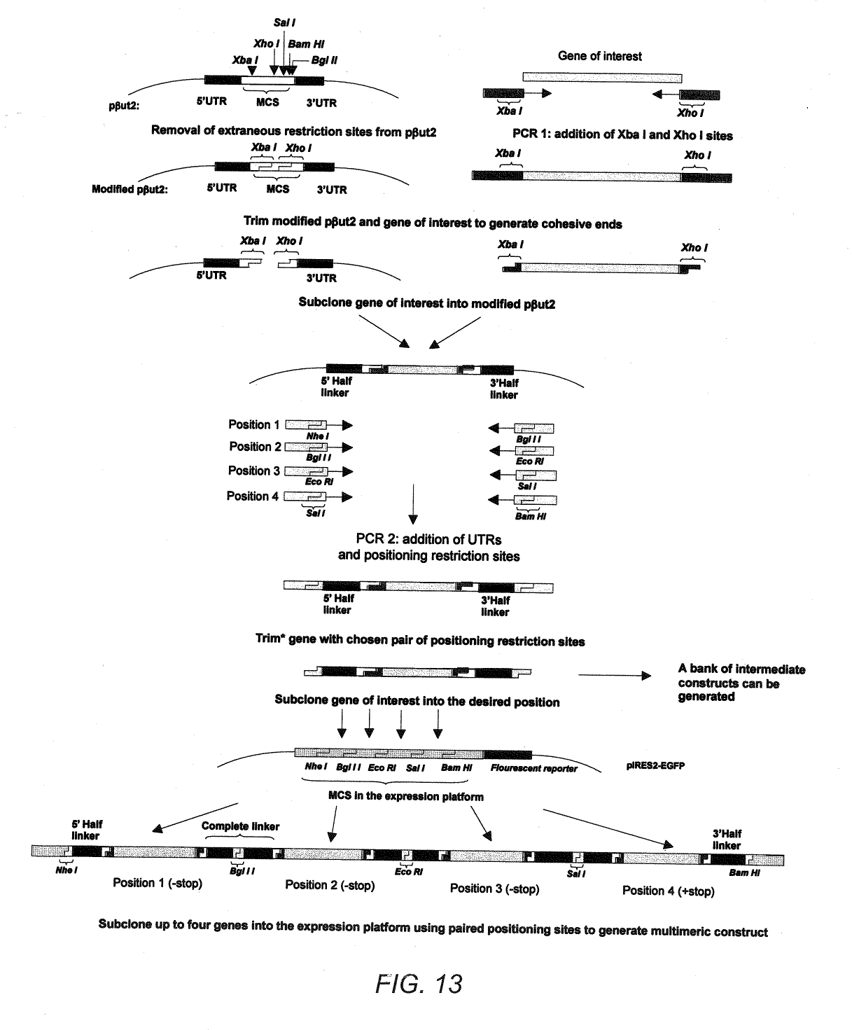

The values (S.E.M.) for activation (A) and inactivation (fast, fI and slow sI) were calculated by fitting to single-and double-exponential functions, respectively.
V½ and slope k were calculated by Boltzmann equation fitting of the gk-V relations from peak outward currents.
IC₅₀ values (±S.E.M.) were derived from the plots exemplified for TEA and AgTX1 in FIG. 12A and 12B.
Values in brackets/parentheses represent the number of experiments.

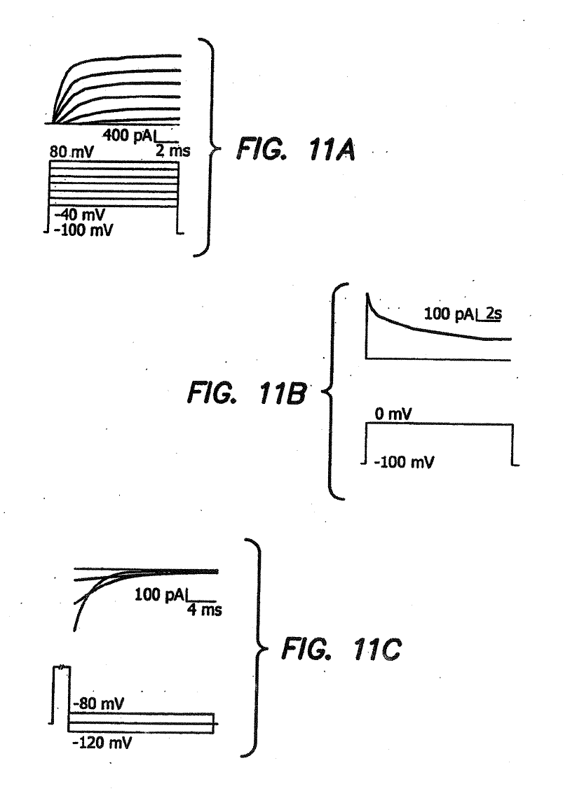

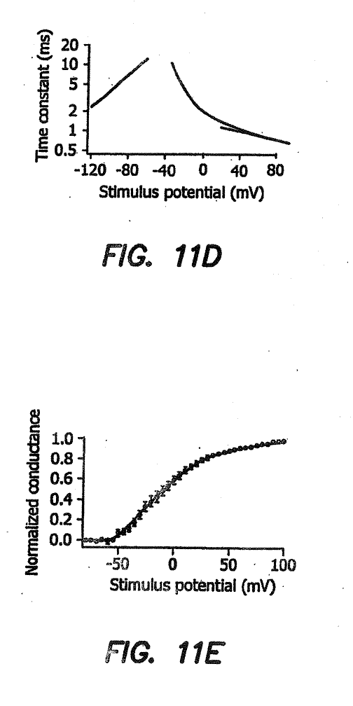

FIGS. 11A-11E show that the adjacent Kv1.1-1.1-1.2-1.2 construct expresses functionally-uniform K⁺ channels. FIGS. 11A-11E are reproduced from FIG. 9 to allow comparison with the other tetrameric channel. FIG. 11A shows the K⁺ currents (I_{K}) from Kv1.1-1.1-1.2-1.2, recorded by conventional patch-clamp, in response to depolarising steps (gray traces) from −40 to +80 mV in +20 mV increments, are consistent with the power of an exponential function (black lines). FIG. 11B shows inactivation of I_{K} from Kv1.1-1.1-1.2-1.2 during a pulse to 0 mV (gray traces), consistent with a double exponential function (black line). FIG. 11C shows deactivation of I_{K} from Kv1.1-1.1-1.2-1.2 at −80, −100 or −120 mV (gray traces) after 50 ms at +60 mV, consistent with mono-exponential function (black lines). FIG. 11D shows time constants (n=3) associated with activation (>−50 mV) or deactivation (<−50 mV) from Kv1.1-1.1-1.2-1.2; lines are consistent with exponential functions. FIG. 11E shows conductance-voltage relationship of outward K⁺ peak currents from Kv1.1-1.1-1.2-1.2 (closed circle; n=15) after 100 ms at indicated voltages; lines are Boltzmann fits. In D and E, error bars represent ±S.D.

FIG. 12A-12C show that adjacently and diagonally-arranged Kv1.1 and 1.2 gene constructs yield channels that can be distinguished by TEA, or AgTX1 and TsTX-Kα. FIG. 12A shows Q Patch voltage-clamp records of the inhibition of K⁺ currents from the channels specified by TEA, AgTX1 or TsTx-Kα. Left panels depict current amplitudes in the absence, presence for the times shown by the bars of different concentrations of the agents, after wash-out of each dose (i) or final removal of blockers (iv). The middle and right panels show representative current traces from adjacently- and diagonally-arranged channels, respectively, in the absence (black line) and presence (grey) of the concentrations shown for each compound.

FIG. 12B shows dose-response curves for the forward Kv1.1-1.1-1.2-1.2 (open circles) and reverse Kv1.2-1.2-1.1-1.1 (closed circles) adjacent channels show a ˜11-fold lower affinity for TEA than the corresponding diagonals (open triangle and closed inverted triangle). (Inset) The channel made using the dimer (Kv1.1-1.2) showed a TEA susceptibility similar to that of both the adjacent arrangements.

FIG. 12C shows dose dependence curves for the sensitivities of adjacent (open circles) and diagonal (closed circles) oligomers to AgTX1; note that the later proved virtually insensitive. The bar diagram shows the inhibition of Iₖ produced by channels containing Kv1.2 (black square), Kv1.2-1.2 (grey square) and Kv1.1-1.2 (open square). All dose response curves were fitted using the Hill equation, where the slopes were between 0.76 and 0.92. The difference in sensitivity to AgTX1 of channels made from Kv1.1-1.2 dimer and Kv1.2 monomer is not statistically significant (p=0.0984 and 0.1321, respectively, unpaired t-test). All the IC₅₀s are listed in Table 2 together with the n values and error bars.

The data from these examples establish the importance of α subunit arrangements within these particular concatamers in governing pharmacological differences and how oligomeric subtypes can be distinguished with appropriate blockers. It will be understood that the present invention is equally applicable to all mammalian, avian, or reptilian neurons, including, without exception, rat, mouse, human, canine, bovine, equine, and feline systems.

The preceding Examples comprise specific embodiments of the present invention, but are not intended to, and do not limit the scope of the invention thereto. The present invention is defined solely by the claims that conclude this specification. Although the foregoing invention has been described in detail for purposes of clarity of understanding, it will be obvious that certain modifications may be practiced within the scope of the appended claims. All publications and patent documents cited herein are hereby incorporated by reference in their entirety for all purposes to the same extent as if each were so individually denoted.

We claim:

1) A method of making a polymeric protein having a N-terminus and a C-terminus and a defined order of specific monomer subunits covalently linked, comprising:
a) amplifying one or more monomer nucleic acid species encoding a monomer subunit species to be contained in the polymeric protein;
b) creating a linker set comprising first, second, third and fourth designated nucleic acid linker pairs; wherein
1) each designated linker pair comprises a 5′ designated linker having a 5′ subcloning restriction endonuclease recognition site different from that of the 5′ designated linker of any other designated linker pair of said linker set;
2) a 3′ designated linker having a 3′ subcloning restriction endonuclease recognition site different from that of any other 3′ designated linker of any other designated linker pair of said linker set;
3) wherein each of the first, second, third, and fourth designated linker pairs is structured to correlate with a first, second, third, and fourth monomer subunit position, respectively, in the tetrameric protein; and
4) wherein
A) the 3′ subcloning restriction endonuclease recognition site of the first designated linker pair is the same as the 5′ subcloning restriction endonuclease recognition site of the second designated linker pair;
B) the 3′ subcloning restriction endonuclease recognition site of the second designated linker pair is the same as the 5′ subcloning restriction endonuclease recognition site of the third designated linker pair;
C) the 3′ subcloning restriction endonuclease recognition site of the third designated linker pair is the same as the 5′ subcloning restriction endonuclease recognition site of the fourth designated linker pair; and
c) individually joining amplified monomer nucleic acid species to 3′ and 5′ designated linkers of each of the first, second, third and fourth designated linker pairs to form a bank of first, second, third, and fourth in-frame nucleic acid monomer species constructs encoding a specific monomer subunit, each said construct having designated restriction site pairs at its 3′ and 5′ end correlating to one of four positions within the polymeric protein,
d) selecting a first, second, third, and fourth nucleic acid monomer species construct encoding the desired monomer subunit for each such position of the polymeric protein,
e) subcloning said first, second, third and fourth nucleic acid monomer species constructs into an expression vector having, in the 5′ to 3′ direction, restriction sites for joining with the 5′ restriction site of each of the first, second, third and fourth nucleic acid monomer species constructs, and a 3′ restriction site for joining with the 3′ end of the fourth nucleic acid monomer species construct, wherein the expression vector is structured to insert each nucleic acid monomer species construct in a common open reading frame, and
f) inserting said expression vector within a suitable host cell,
g) expressing within said host a polypeptide comprising a polymeric protein comprising a defined order of specific monomer subunits.

2. The method of claim 1 further comprising between steps a) and b) the steps:
A1) attaching to the 5′ end of each one or more monomer nucleic acid species a first cloning linker comprising a first cloning restriction endonuclease recognition site; and
A2) attaching to the 3′ end of each one or more monomer nucleic acid species a second cloning linker comprising a second cloning restriction endonuclease recognition site;
A3) cleaving each said one or more monomer nucleic acid species with said first and second restriction endonuclease; and
A4) joining each said monomer nucleic acid species with a first subcloning vector also cleaved with said first and second restriction endonuclease.

3) The method of claim 1 wherein at least one of the first, second, third and fourth designated linker pairs also contain an untranslated region of a Xenopus β-globin gene.

4) The method of claim 3 wherein each of the first, second, third and fourth designated linker pairs also contain an untranslated region of a Xenopus β-globin gene.

5) The method of claim 1 wherein the expression vector also contains a co-transcribed florescent protein tag for expression with and as part of the expressed polypeptide.

6) The method of claim 1 wherein the expression vector contains a co-transcribed green fluorescent protein tag for expression with the expressed polypeptide.

7) The method of claim 1 wherein the collection of different monomer subunit species comprises potassium channel α subunit species.

8) The method of claim 1 wherein the collection of different monomer subunit species comprises potassium channel α subunit species.

9) The method of claim 1 wherein the collection of different monomer subunit species comprises potassium channel αKv1.x subunit species.

10) A collection of nucleic acids comprising one or more nucleic acid monomer subunit species separately joined to each of first, second, third and fourth designated nucleic acid linker pairs of to make first, second, third, and fourth nucleic acid monomer subunit subspecies, wherein each designated nucleic acid linker pair comprises a 3′ subcloning restriction endonuclease recognition site and a 5′ subcloning restriction endonuclease recognition site, wherein;
a) the 3′ subcloning restriction endonuclease recognition site of the first designated linker pair is the same as the 5′ subcloning restriction endonuclease recognition site of the second designated linker pair;
b) the 3′ subcloning restriction endonuclease recognition site of the second designated linker pair is the same as the 5′ subcloning restriction endonuclease recognition site of the third designated linker pair;
c) the 3′ subcloning restriction endonuclease recognition site of the third designated linker pair is the same as the 5′ subcloning restriction endonuclease recognition site of the fourth designated linker pair; and wherein the first, second, third, and fourth nucleic acid monomer subunit subspecies are structured to maintain a common open reading frame when ligated together in the sequence, in the direction from 5′ to 3′, first, second, third, and fourth nucleic acid monomer subunit subspecies.

11) The collection of claim 10 comprising more than one nucleic acid monomer subunit species.

12) The collection of claim 10, wherein the fourth nucleic acid monomer subunit subspecies comprises an in-frame stop codon.

13) The collection of claim 10 comprising nucleic acid monomer subunit species encoding potassium channel α subunit monomers.

14) The collection of claim 10 comprising nucleic acid monomer subunit species encoding potassium channel αKv1.x subunit species.

15) A kit comprising a cassette cloning system for the expression of a concatenated multisubunit protein comprising;
a) a nucleic acid expression vector comprising a multiple cloning site having at least a first restriction nuclease recognition site and a second different nucleic acid recognition site located to the 3′ of the first site; and
b) a nucleic acid collection comprising one or more nucleic acid monomer subunit species separately joined to each of first, second, third and fourth designated nucleic acid linker pairs of to make first, second, third, and fourth nucleic acid monomer subunit subspecies, wherein each designated nucleic acid linker pair comprises a 3′ subcloning restriction endonuclease recognition site and a 5′ subcloning restriction endonuclease recognition site, wherein;
a) the 3′ subcloning restriction endonuclease recognition site of the first designated linker pair is the same as the 5′ subcloning restriction endonuclease recognition site of the second designated linker pair;
b) the 3′ subcloning restriction endonuclease recognition site of the second designated linker pair is the same as the 5′ subcloning restriction endonuclease recognition site of the third designated linker pair;
c) the 3′ subcloning restriction endonuclease recognition site of the third designated linker pair is the same as the 5′ subcloning restriction endonuclease recognition site of the fourth designated linker pair; and wherein the first, second, third, and fourth nucleic acid monomer subunit subspecies are structured to maintain a common open reading frame when ligated together in the sequence, in the direction from 5′ to 3′, first, second, third, and fourth nucleic acid monomer subunit subspecies,
wherein the 5′ subcloning restriction endonuclease recognition site of one nucleic acid monomer subunit subspecies is the same as the first vector restriction endonuclease recognition site, and the 3′ subcloning restriction endonuclease recognition site of another nucleic acid monomer subunit subspecies is the same as the second vector restriction endonuclease recognition site.

16) The kit of claim 15 comprising nucleic acid monomer subunit species encoding potassium channel α subunit monomers.

17) The kit of claim 16 comprising nucleic acid monomer subunit subspecies encoding potassium channel αKv1.x subunit species.

18) The kit of claim 15, wherein the fourth nucleic acid monomer subunit subspecies comprises an in-frame stop codon.

19) The kit of claim 15 wherein the expression vector is a bacterial expression vector.

20) The kit of claim 15 wherein the expression vector contains a nucleic acid sequence encoding a florescent protein.
